(19) United States
(12) Patent Application Publication
(10) Pub. No.: US 2005/0186600 A1
(43) Pub. Date: Aug. 25, 2005

(54) Polynucleotides encoding novel UbcH10 polypeptides and kits and methods using same

(76) Inventors: Osnat Sella-Tavor, Kfar-Kish (IL); Galit Rotman, Herzlia (IL); Sarah Pollock, Tel-Aviv (IL); Alex Diber, Rishon-LeZion (IL); Shira Walach, Hod-HaSharon (IL); Shirley Sameach-Greenwald, Kfar-Saba (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 11/034,010

(22) Filed: Jan. 13, 2005

Related U.S. Application Data
(60) Provisional application No. 60/535,904, filed on Jan. 13, 2004.
(60) Provisional application No. 60/572,122, filed on May 19, 2004.

(51) Int. Cl. C12Q 1/68; C07H 21/04; C12N 9/10; C12N 9/64

(52) U.S. Cl. 435/6; 435/69.1; 435/226; 435/320.1; 435/325; 536/23.2

(57) ABSTRACT

An isolated polynucleotide is provided. The isolated polynucleotide comprising a nucleic acid sequence encoding a UbcH10 polypeptide having at least a portion of an amino acid sequence at least 55% homologous to SEQ ID NO:7, as determined using the BlastP software of the National Center of Biotechnology Information (NCBI) using default parameters.

This patent application claims priority from and is related to U.S. Provisional Patent Applications Ser. No. 60/535,904, filed 13 Jan. 2004 and Ser. No. 60/572,122, filed 19 May 2004, these two U.S. Provisional patent applications incorporated by reference in their entirety herein.

### BACKGROUND OF THE INVENTION

The present invention relates to novel UbcH10 polypeptides and polynucleotides encoding same. More particularly, the present invention relates to methods and kits for diagnosing and treating UbcH10-related diseases, such as cancer.

### FIELD OF THE INVENTION

Ubiquitination is the most widely employed proteolytic mechanism in eukaryotic cells. Ubiquitination involves the covalent modification of proteins with ubiquitin, a highly conserved 76 amino acid protein. The covalent attachment of ubiquitin to the substrates follows a reaction mechanism involving the sequential action of three enzymes, which are termed E1, E2 and E3 and marks the substrate for degradation by the 26S proteosome.

The ubiquitin system plays key roles in every aspect of biology, including cell growth, cell cycle, apoptosis, signal transduction, DNA repair, transcription, antigen processing and ion-channel regulation. Not surprisingly, the disregulation of ubiquitin-mediated process has been implicated in several diseases, including cancer.

As mentioned, ubiquitin is linked to protein substrates in a reaction mechanism involving the sequential action of three enzymes; Ubiquitin is first activated in an ATP dependent manner by a ubiquitin-activating enzyme, called E1. Activated ubiquitin is then transferred via a thiolester intermediate to a ubiquitin-conjugating enzyme, called E2. This activated E2 then acts in concert with a ubiquitin-ligase, called E3, to transfer the ubiquitin to a target substrate, forming an isopeptide bond between the ε-amino group of the substrate's Lys residue and the C-terminal Gly residue of ubiquitin. The E3 reaction is repeated such that a chain of ubiquitin molecules (i.e., polyubiquitin) is attached to the protein. Poly-ubiquitinated proteins can then be recognized and degraded by the 26S proteosome.

It is now appreciated that the basic components of the ubiquitin system which were first characterized (ubiquitin, E1, E2 and E3), are founding members of much larger gene families or functional groups, which are now generically referred to the E1, E2 and E3 classes. Most organisms have a single E1, and several E2 enzymes. The yeast genome, for example, codes for 13 E2 enzymes. The E3 family, which is responsible for the substrate specificity of the reaction, is the most populous. E3s interact specifically with both the E2 enzymes and protein substrates. E3s therefore regulate ubiquitination by bringing substrates together with the rest of the ubiquitination machinery. The expansion of the E2 and E3 families suggests that protein degradation is regulated at the level of the ubiquitination machinery.

Accumulating evidence implicate ubiquitin components in disease onset and progression, such as cancer. For example, various reports have suggested that E3 proteins play important roles in the regulation of oncoproteins, including p53, c-jun, β-catenin, VHL, c-cbl and, recently, hCdc4. Oncogenic strains of papilloma virus, which harbor a particular sequence variant of the Papilloma virus E6 protein, target p53 for degradation. The E6 variant forms a complex with p53 as well as an E2/E3 complex (UbcH8 and E6-AP respectively); in effect, the E6 protein bridges the proteolytic machinery and the p53 substrate. The intracellular levels of c-jun are also regulated in part through proteolysis, as a domain within c-jun that is responsible for its ubiquitination, is absent from its oncogenic variant v-jun4. The c-cbl protooncogene is in fact an E3 protein that targets several receptor tyrosine kinases for ubiquitin-mediated proteolysis. β-catenin, which is implicated in many forms of cancer, binds and activates several transcription factors. The availability of β-catenin is kept low by phosphorylation dependent proteolysis.

The relationship of proteolysis to cancer also extends to transcription and cell cycle. The E3 component Skp2 mediates the cell-cycle-dependent degradation of p27, an inhibitor of several cell-cycle kinases. Several studies implicate increased Skp2 and decreased p27 levels in cancer progression. The human orthologue of the yeast cdc4 protein is part of an E3 complex that degrades cyclin E, and which has recently been shown to be mutated in breast cancer.

Altogether, the above-findings attribute a role for the ubiquitin pathway in general and the E3 family of proteins, in particular, in cell cycle progression and tumor cell growth.

The E2 component of the ubiquitin pathway includes a plurality of genes encoding structurally related proteins which share a conserved domain of 16,000 dalton which includes a cysteine residue that is required for the formation of ubiquitin-E2 thiol ester [Ciechanover (1994) Cell 79:13-21]. A number of reports associate E2 family members with cell-cycle progression and tumorogenesis. For example, it has been shown that overexpression of Ubc2/Rad6 induces anchorage-independent growth of recipient cells, indicating that deregulated expression of Ubc2/Rad6 is involved in malignant transformation [Shekhar (2002) Cancer Res. 62:2115-2124]. Indeed, Ubc2/Rad6 and Ubc3/CDC34 were shown to be specifically involved in the Ubiquitin-dependent degradation of cylcin-dependent kinase inhibitor p27 [Pagano (1995) Science 269:682-685]. Ubc9 is another example for the involvement of E2s in tumorogenesis, as expression levels of Ubc9 were increased in human lung adenocarcinomas compared to normal lung tissue [McDoniels-Silvers (2002) Clin. Cancer Res. 8:1127-1138]. Finally, inactive forms of E2, termed UEV, which share structural homology to the E2 however lacking enzymatic activity are found to be down-regulated in colon carcinoma cell lines and in prostate cancer [Sancho (1998) Mol. Cell Biol. 18(1): 576-89; Stubbs (1999) Am. J. Pathol. 154:1335-43].

UbcH10 was cloned as the human homologue of the cyclin-selective E2 (E2-C) enzyme shown to mediate the degradation of mitotic cyclins [Townsley (1997) Proc. Natl. Acad. Sci. USA 94:2362-2367; King (1995) Cell 81:279-288; Aristarkhov (1996) Proc. Natl. Acad. Sci. USA 93:4294-4299; Townsley (1997) Proc. Natl. Acad. Sci. USA 94:2362-2367]. The expression of UbcH10 is cell-cycle regulated; it is highly expressed in G2-M phase, while hardly any expression of this protein is detected in the G0-G1 phase. These findings suggest that the function of UbcH10 is closely related to cell cycle progression [Townsley (1997) Supra] and to tumor onset and progression.

Recently, it was uncovered that UbcH10 expression is upregulated in NIH3T3 cells transformed with EWS/FLI1 but not in non-transformed cells, indicating that UbcH10 also plays an important role in cellular transformation. These findings were substantiated by a study that illustrated that UbcH10-expressing NIH3T3 cells are able to form colonies in soft-agar. These findings were further established by Okamoto and co-workers [Okamoto (2003) Cancer Res. 63:4167-4173] who showed elevated levels of wild-type UbcH10 (GenBank Accession No. U73379) in cancerous tissues of the lung, stomach, uterus and bladder.

### SUMMARY OF THE INVENTION

The background art fails to teach novel naturally occurring variants of UbcH10 which are overexpressed in cancer and can be used to diagnose predisposition to, prognosis, prediction, screening, early diagnosis, staging, therapy selection, treatment monitoring and facilitate design of therapeutic tools for, UbcH10 related diseases, such as cancers.

The present invention overcomes the deficiencies of the background art by providing novel UbcH10 transcripts and polypeptides which can be used to diagnose and treat UBCH10-related diseases such as cancer. According to a preferred embodiment of the present invention, these transcripts and polypeptides may optionally be used as novel markers for UbcH10 related cancers that are both sensitive and accurate. These markers are overexpressed in UbcH10 related cancers specifically, as opposed to normal tissues. The measurement of these markers, alone or in combination, in patient samples provides information that the diagnostician can correlate with a probable diagnosis of UbcH10 related cancer. The markers of the present invention, alone or in combination, show a high degree of differential detection between cancerous and non-cancerous states.

According to one aspect of the present invention there is provided an isolated polynucleotide comprising a nucleic acid sequence encoding a UbcH10 polypeptide having at least a portion of an amino acid sequence at least 55% homologous to SEQ ID NO:7, as determined using the BlastP software of the National Center of Biotechnology Information (NCBI) using default parameters.

According to another aspect of the present invention there is provided an isolated polynucleotide comprising a nucleic acid sequence encoding a UbcH10 polypeptide having at least a portion of an amino acid sequence at least 70% homologous to SEQ ID NO:7, as determined using the BlastP software of the National Center of Biotechnology Information (NCBI) using default parameters.

According to yet another aspect of the present invention there is provided an isolated polynucleotide including a nucleic acid sequence at least 60% identical to SEQ ID NO:1, 2 or 3, as determined using the BlastN software of the National Center of Biotechnology Information (NCBI) using default parameters.

According to yet another aspect of the present invention there is provided an isolated polynucleotide as set forth by SEQ ID NO:1, 2 or 3.

According to still another aspect of the present invention there is provided an isolated polypeptide encoding for UbcH10, comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order.

According to an additional aspect of the present invention there is provided an antibody or an antibody fragment being capable of specifically binding an isolated polypeptide encoding for UbcH10, wherein the isolated polypeptide comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order.

According to yet an additional aspect of the present invention there is provided an isolated polypeptide encoding for UbcH10, comprising a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order.

According to still an additional aspect of the present invention there is provided an antibody or an antibody fragment being capable of specifically binding an isolated polypeptide encoding for UbcH10, wherein the isolated polypeptide comprises a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order.

According to a further aspect of the present invention there is provided an isolated polypeptide encoding for UbcH10, comprising a first amino acid sequence being at least 90% homologous to amino acids 1-72 as set forth in SEQ ID NO:11, and a second amino acid sequence being at least 80% homologous to amino acid sequence as set forth of SEQ ID NO:8, wherein the first amino acid and the second amino acid sequence are contiguous and in a sequential order.

According to yet a further aspect of the present invention there is provided an antibody or an antibody fragment being capable of specifically binding an isolated polypeptide encoding for UbcH10, wherein the isolated polypeptide comprises a first amino acid sequence being at least 90% homologous to amino acids 1-72 as set forth in SEQ ID NO:11, and a second amino acid sequence being at least 80% homologous to amino acid sequence as set forth of SEQ ID NO:8, wherein the first and the second amino acid sequences are contiguous and in a sequential order.

According to still a further aspect of the present invention there is provided an antibody or an antibody fragment being capable of specifically binding a UbcH10 polypeptide including at least a portion of an amino acid sequence at least 55% homologous to SEQ ID NO:4, 5, 6, or 7, as determined using the BlastP software of the National Center of Biotechnology Information (NCBI) using default parameters.

According to still a further aspect of the present invention there is provided an antibody or an antibody fragment being capable of specifically binding a UbcH10 polypeptide including at least a portion of an amino acid sequence at least 55% homologous to SEQ ID NO:7, as determined using the BlastP software of the National Center of Biotechnology Information (NCBI) using default parameters.

According to still a further aspect of the present invention there is provided an antibody or an antibody fragment being capable of specifically binding a UbcH10 polypeptide including at least a portion of an amino acid sequence at least 55% homologous to SEQ ID NO:8, as determined using the BlastP software of the National Center of Biotechnology Information (NCBI) using default parameters.

According to still a further aspect of the present invention there is provided a display library comprising a plurality of display vehicles each displaying at least 6 consecutive amino acids derived from a UbcH10 polypeptide including at least a portion of an amino acid sequence at least 55% homologous to SEQ ID NO:7 or 8, as determined using the BlastP software of the National Center of Biotechnology Information (NCBI) using default parameters.

According to still a further aspect of the present invention there is provided an oligonucleotide specifically hybridizable with a nucleic acid sequence encoding a UbcH10 polypeptide including at least a portion of an amino acid sequence at least 55% homologous to SEQ ID NO:7 or 8, as determined using the BlastP software of the National Center of Biotechnology Information (NCBI) using default parameters.

According to still a further aspect of the present invention there is provided a pharmaceutical composition comprising a therapeutically effective amount of a UbcH10 polypeptide including at least a portion of an amino acid sequence at least 55% homologous to SEQ ID NO:7 or 8, as determined using the BlastP software of the National Center of Biotechnology Information (NCBI) using default parameters.

According to still a further aspect of the present invention there is provided a method of diagnosing predisposition to, or presence of a UbcH10-related disease in a subject, the method comprising determining a level of a UbcH10 polypeptide including at least a portion of an amino acid sequence at least 55% homologous to SEQ ID NO:7 or 8, as determined using the BlastP software of the National Center of Biotechnology Information (NCBI) using default parameters, or of a polynucleotide encoding the polypeptide in a biological sample obtained from the subject, wherein the level of the polynucleotide or the level of the polypeptide is correlatable with predisposition to, or presence or absence of the UbcH10-related disease, thereby diagnosing predisposition to, or presence of UbcH10-related disease in the subject.

According to still a further aspect of the present invention there is provided a method of diagnosing predisposition to, or presence of a UbcH10-related disease in a subject, the method comprising determining a level of a UbcH10 polypeptide including at least a portion of an amino acid sequence at least 70% homologous to a UbcH10 polypeptide as set forth in SEQ ID NO:4, 5 or 6, as determined using the BlastP software of the National Center of Biotechnology Information (NCBI) using default parameters, or of a polynucleotide encoding the polypeptide in a biological sample obtained from the subject being at least 70% identical to a polynucleotide as set forth by SEQ ID NO:1, 2 or 3, wherein said level of the polynucleotide or the level of the polypeptide is correlatable with predisposition to, or presence or absence of the UbcH10-related disease, thereby diagnosing predisposition to, or presence of UbcH10-related disease in the subject.

According to still a further aspect of the present invention there is provided a use of a UbcH10 polypeptide including at least a portion of an amino acid sequence at least 70% homologous to a UbcH10 polypeptide as set forth in SEQ ID NO:4, 5 or 6, as determined using the BlastP software of the National Center of Biotechnology Information (NCBI) using default parameters, or of a polynucleotide encoding the polypeptide, the polynucleotide being at least 70% identical to a polynucleotide as set forth by SEQ ID NO:1, 2 or 3, in the manufacturing of a diagnostic agent suitable for determining predisposition to, or presence of a UbcH10-related disease in a subject.

According to still a further aspect of the present invention there is provided a method of treating UbcH10-related disease in a subject, the method comprising specifically upregulating in the subject expression of a UbcH10 polypeptide at least 55% homologous to SEQ ID NO:7 or 8, as determined using the BlastP software of the National Center of Biotechnology Information (NCBI) using default parameters.

According to still a further aspect of the present invention there is provided a method of treating UbcH10-related disease in a subject, the method comprising specifically downregulating in the subject expression level and/or activity of a UbcH10 polypeptide at least 55% homologous to SEQ ID NO:7 or 8, as determined using the BlastP software of the National Center of Biotechnology Information (NCBI) using default parameters.

According to still a further aspect of the present invention there is provided a kit for diagnosing UbcH10-related disease or a predisposition thereto in a subject, the kit comprising at least one reagent capable of detecting overexpression of at least one isolated polypeptide encoding for UbcH10 selected from the group consisting of an isolated polypeptide comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-72 as set forth in SEQ ID NO:11, and a second amino acid sequence being at least 80% homologous to amino acid sequence as set forth of SEQ ID NO:8, wherein the first amino acid and the second amino acid sequence are contiguous and in a sequential order, and an isolated polypeptide as set forth by SEQ ID NO:4, 5, 6, 7 or 8.

According to still a further aspect of the present invention there is provided a method of diagnosing predisposition to, or presence of a UbcH10-related disease in a subject, the method comprising determining a level of at least one isolated polypeptide encoding for UbcH10 selected from the group consisting of an isolated polypeptide comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-72 as set forth in SEQ ID NO:11, and a second amino acid sequence being at least 80% homologous to amino acid sequence as set forth of SEQ ID NO:8, wherein the first and second amino acid sequences are contiguous and in a sequential order, and an isolated polypeptide as set forth by SEQ ID NO:4, 5, 6, 7 or 8.

According to further features in preferred embodiments of the invention described below, the UbcH10 polypeptide is as set forth in SEQ ID NO:4, 5, or 6.

According to still further features in the described preferred embodiments the nucleic acid sequence is as set forth in SEQ ID NO:1 or 2.

According to still further features in the described preferred embodiments the isolated polypeptide is set forth by SEQ ID NO:4.

According to still further features in the described preferred embodiments the edge polypeptide is set forth by SEQ ID NO:7.

According to still further features in the described preferred embodiments the edge polypeptide includes at least one bridge portion.

According to still further features in the described preferred embodiments the at least one bridge portion includes a first bridge portion and a second bridge portion.

According to still further features in the described preferred embodiments the first bridge portion comprises a polypeptide having “n” amino acids, wherein the “n” is at least 10 and whereas at least two amino acids of the first bridge portion are Threonine and Alanine, and wherein the first bridge portion has a structure as follows (numbering according to SEQ ID NO:4): a sequence starting from any of amino acid numbers 72−x to 72; and ending at any of amino acid numbers 73+((n−2)−x), in which x varies from 0 to n−2.

According to still further features in the described preferred embodiments the first bridge portion comprises a polypeptide having “n” amino acids, wherein the “n” is at least 4 and whereas at least two amino acids of the first bridge portion are Threonine and Alanine, and wherein the first bridge portion has a structure as follows (numbering according to SEQ ID NO:4): a sequence starting from any of amino acid numbers 72−x to 72; and ending at any of amino acid numbers 73+((n−2)−x), in which x varies from 0 to n−2.

According to still further features in the described preferred embodiments the second bridge portion comprises a polypeptide having “n” amino acids, wherein the “n is at least 10, and whereas at least two amino acids of the second bridge portion are Proline and Glutamic acid, and wherein the second bridge portion has a structure as follows (numbering according to SEQ ID NO:4): a sequence starting from any of amino acid numbers 122−x to 122; and ending at any of amino acid numbers 123+((n−2)−x), in which x varies from 0 to n−2.

According to still further features in the described preferred embodiments the second bridge portion comprises a polypeptide having “n” amino acids, wherein the “n is at least 4, and whereas at least two amino acids of the second bridge portion are Proline and Glutamic acid, and wherein the second bridge portion has a structure as follows (numbering according to SEQ ID NO:4): a sequence starting from any of amino acid numbers 122−x to 122; and ending at any of amino acid numbers 123+((n−2)−x), in which x varies from 0 to n−2.

According to still further features in the described preferred embodiments the isolated polypeptide is set forth by SEQ ID NO:5.

According to still further features in the described preferred embodiments the first bridge portion comprises a polypeptide having “n” amino acids, wherein the “n” is at least 10 and whereas at least two amino acids of the first bridge portion are Methionine and Alanine, and wherein the first bridge portion has a structure as follows (numbering according to SEQ ID NO:5): a sequence starting from any of amino acid numbers 42−x to 42; and ending at any of amino acid numbers 43+((n−2)−x), in which x varies from 0 to n−2.

According to still further features in the described preferred embodiments the first bridge portion comprising a polypeptide having “n” amino acids, wherein the “n” is at least 4 and whereas at least two amino acids of the first bridge portion are Methionine and Alanine, and wherein the first bridge portion has a structure as follows (numbering according to SEQ ID NO:5): a sequence starting from any of amino acid numbers 42−x to 42; and ending at any of amino acid numbers 43+((n−2)−x), in which x varies from 0 to n−2.

According to still further features in the described preferred embodiments the second bridge portion comprises a polypeptide having “n” amino acids, wherein the “n” is at least 10, and whereas at least two amino acids of the second bridge portion are Proline and Glutamic acid, and wherein the second bridge portion has a structure as follows (numbering according to SEQ ID NO:5): a sequence starting from any of amino acid numbers 93−x to 93; and ending at any of amino acid numbers 94+((n−2)−x), in which x varies from 0 to n−2.

According to still further features in the described preferred embodiments the second bridge portion comprises a polypeptide having “n” amino acids, wherein the “n” is at least 4, and whereas at least two amino acids of the second bridge portion are Proline and Glutamic acid, and wherein the second bridge portion has a structure as follows (numbering according to SEQ ID NO:5): a sequence starting from any of amino acid numbers 93−x to 93; and ending at any of amino acid numbers 94+((n−2)−x), in which x varies from 0 to n−2.

According to still further features in the described preferred embodiments the isolated polypeptide is set forth by SEQ ID NO:6.

According to still further features in the described preferred embodiments a bridge portion between the first amino acid sequence and the second amino acid sequence is a polypeptide having “n” amino acids, wherein the “n” is at least 10 and whereas at least two amino acids of the bridge portion are Threonine and Arginine, and wherein the bridge portion has a structure as follows (numbering according to SEQ ID NO:6): a sequence starting from any of amino acid numbers 72−x to 72; and ending at any of amino acid numbers 73+((n−2)−x), in which x varies from 0 to n−2 such that the value ((n−2)−x) is not allowed to be larger than 4.

According to still further features in the described preferred embodiments the polypeptide is as set forth by SEQ ID NO:4, 5, 6 or 7.

According to still further features in the described preferred embodiments the polypeptide is as set forth in SEQ ID NO:8.

According to still further features in the described preferred embodiments the nucleic acid sequence is as set forth in SEQ ID NO:1, 2 or 3.

According to still further features in the described preferred embodiments the oligonucleotide is a single or double stranded.

According to still further features in the described preferred embodiments the oligonucleotide is at least 10 bases long.

According to still further features in the described preferred embodiments the oligonucleotide is hybridizable in either sense or antisense orientation.

According to still further features in the described preferred embodiments the UbcH10-related disease is selected from the group consisting of ovarian cancer and lung cancer.

According to still further features in the described preferred embodiments determining level of the polypeptide is effected via an assay selected from the group consisting of immunohistochemistry, ELISA, RIA, Western blot analysis, FACS analysis, an immunofluorescence assay, and a light emission immunoassay.

According to still further features in the described preferred embodiments determining level of the polynucleotide is effected via an assay selected from the group consisting of PCR, RT-PCR, quantitative RT-PCR, chip hybridization, RNase protection, in-situ hybridization, primer extension, Southern blot, Northern blot and dot blot analysis.

According to still further features in the described preferred embodiments the polynucleotide is as set forth by SEQ ID NO:1, 2 or 3.

According to still further features in the described preferred embodiments upregulating expression of the polypeptide is effected by: (i) administering the polypeptide to the subject; (ii) administering an expressible polynucleotide encoding the polypeptide to the subject; (iii) increasing the endogenous level of UbcH10 polypeptide in the subject; (iv) increasing the endogenous activity of UbcH10 polypeptide in the subject; (v) introducing at least one substrate of UbcH10 polypeptide to the subject; and/or (vi) administering UbcH10 polypeptide-expressing cells into the subject.

According to still further features in the described preferred embodiments downregulating is effected by introducing into the subject an agent selected from the group consisting of: (a) a molecule which binds the UbcH10 polypeptide; (b) an enzyme which cleaves the UbcH10 polypeptide; (c) an antisense polynucleotide capable of specifically hybridizing with at least part of an mRNA transcript encoding the UbcH10 polypeptide; (d) a ribozyme which specifically cleaves at least part of an mRNA transcript encoding the UbcH10 polypeptide; (e) a small interfering RNA (siRNA) molecule which specifically cleaves at least part of a transcript encoding the UbcH10 polypeptide; (f) a non-functional analogue of at least a catalytic or binding portion of the UbcH10 polypeptide; (g) a molecule which prevents the UbcH10 polypeptide activation or substrate binding.

According to still further features in the described preferred embodiments the at least one reagent is an antibody or antibody fragment

According to still further features in the described preferred embodiments detecting is effected using an assay selected from the group consisting of immunohistochemistry, ELISA, RIA, Western blot analysis, FACS analysis, an immunofluorescence assay, and a light emission immunoassay.

According to still further features in the described preferred embodiments the antibody or antibody fragment is coupled to an enzyme.

According to still further features in the described preferred embodiments the antibody or antibody fragment is coupled to a detectable moiety selected from the group consisting of a chromogenic moiety, a fluorogenic moiety, a radioactive moiety and a light-emitting moiety.

According to still further features in the described preferred embodiments detecting is effected using a NAT-based technology.

According to still further features in the described preferred embodiments the at least one reagent is at least one primer pair capable of selectively hybridizing to a nucleic acid sequence encoding at least one isolated polypeptide encoding for UbcH10 selected from the group consisting of an isolated polypeptide comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-72 as set forth in SEQ ID NO:1, and a second amino acid sequence being at least 80% homologous to amino acid sequence as set forth of SEQ ID NO:8, wherein the first and second amino acid sequences are contiguous and in a sequential order, and an isolated polypeptide as set forth by SEQ ID NO:4, 5, 6, 7 or 8.

According to still further features in the described preferred embodiments the at least one reagent is at least one oligonucleotide capable of selectively hybridizing to a nucleic acid sequence encoding at least one isolated polypeptide encoding for UbcH10 selected from the group consisting of an isolated polypeptide comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:1, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-72 as set forth in SEQ ID NO:1, and a second amino acid sequence being at least 80% homologous to amino acid sequence as set forth of SEQ ID NO:8, wherein the first and second amino acid sequences are contiguous and in a sequential order, and an isolated polypeptide as set forth by SEQ ID NO:4, 5, 6, 7 or 8.

According to still further features in the described preferred embodiments the immunoassay is effected using an antibody selected capable of differentially binding to at least one isolated polypeptide encoding for UbcH10 selected from the group consisting of an isolated polypeptide comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-43 as set forth in SEQ ID NO:1, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order, an isolated polypeptide comprising a first amino acid sequence being at least 90% homologous to amino acids 1-72 as set forth in SEQ ID NO:11, and a second amino acid sequence being at least 80% homologous to amino acid sequence as set forth of SEQ ID NO:8, wherein the first amino acid and the second amino acid sequence are contiguous and in a sequential order, and an isolated polypeptide as set forth by SEQ ID NO:4, 5, 6, 7 or 8.

The present invention successfully addresses the shortcomings of the presently known configurations by providing polynucleotides and polypeptides for diagnosing UbcH10-related diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c present the nucleic acid sequences of the novel UbcH10 variants of the present invention. FIG. 1a—illustrates the nucleic acid sequence of variant as depicted in SEQ ID NO:1; FIG. 1b—illustrates the nucleic acid sequence of variant as depicted in SEQ ID NO:2; FIG. 1c—illustrates the nucleic acid sequence of variant as depicted in SEQ ID NO:3; Start codons and termination codons are highlighted.

FIGS. 2a-c present the amino acid sequences of the novel UbcH10 variants of the present invention. FIG. 2a—the amino acid sequence of UbcH10 variant encoded by SEQ ID NO:1 (SEQ ID NO:4); FIG. 2b—the amino acid sequence of UbcH10 variant encoded by SEQ ID NO:2 (SEQ ID NO:5); FIG. 2c—the amino acid sequence of UbcH10 variant encoded by SEQ ID NO:3 (SEQ ID NO:6); Functional domains are highlighted as follows: Pink—putative E3-APC interacting sites; Green—putative E1 interacting sites [Jiang and Basavappa (1999) Biochemistry 38:6471-78]; Light blue—amino terminal extension; Yellow—unique amino acid sequence.

FIG. 3 is a schematic illustration showing the domain structure of wild-type UbcH10 (GenBank Accession Nos. 000762 and UBCC_HUMAN/; SEQ ID NO:1) as well as of new variants of the present invention, as depicted in SEQ ID NOs:4, 5 and 6. Functional domains are highlighted as follows: Red—catalytic cysteine residue at the active site [Townsley (1997) Proc. Natl. Acad. Sci. USA 94:2362-7; Lin (2002) J. Biol. Chem. 277:21913-21]; Light green—UBC active site [PROSITE-PS00183; Lin (2002) J. Biol. Chem. 277:21913-21]; Grey—UBC family profile—UBC domain [PROSITE-PS50127; Stefan (1992) J., Annu. Rev. Genet. 26:179-207]; Pink—putative E3-APC interacting sites; Green—putative E1 interacting sites [Jiang and Basavappa (1999) Biochemistry 38:6471-78]; Blue—destruction box [Yamanaka (2000) Mol.Biol. Cell 11 2821-31; Lin (2002) Supra]; Purple—amino terminal extension; Yellow—unique amino acid sequence.

FIG. 4 is an illustration showing schematic alignment of the nucleic acid sequences of wild type UbcH10 transcript (GenBank Accession No. U73379; SEQ ID NO:36) and new variants of the present invention, as depicted in SEQ ID NOs:1, 2 and 3. Coding regions are marked in green. Sequence region 4a+4b encode the unique amino acid sequence SEQ ID NO:7, and sequence region 4b encodes the unique amino acid sequence SEQ ID NO:8 in transcript of SEQ ID NO:3 containing same and is marked by diagonal stripes. Red arrows indicate the location of the primers and SEQ ID NOs. thereof, which were used for real-time PCR validation.

FIGS. 5a-b are a histogram (FIG. 5a) and a scatter plot (FIG. 5b) showing the relative expression of UbcH10 variants (e.g., variant as depicted in SEQ ID NO:1) in normal and tumor derived lung samples as determined by real time PCR using primers (SEQ ID NOs:15 and 16) for amplicon as depicted in SEQ ID NO:12. Expression was normalized to the averaged expression of four housekeeping genes PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), Ubiquitin (GenBank Accession No. BC000449; amplicon—SEQ ID NO:33; forward primer—SEQ ID NO:34; reverse primer—SEQ ID NO:35) and SDHA (GenBank Accession No. NM_{—}004168; amplicon—SEQ ID NO:30; forward primer—SEQ ID NO:31; reverse primer—SEQ ID NO:32). Note, the number of samples that showed at least 5 fold over-expression, out of the total number of tested samples, is indicated in FIG. 5a below each cancer subtype.

FIGS. 6a-b are a histogram (FIG. 6a) and a scatter plot (FIG. 6b) showing the relative expression of UbcH10 variants (e.g., variant as depicted in SEQ ID NO:1) in normal, benign and tumor derived ovarian samples as determined by real time PCR using primers (SEQ ID NOs:15 and 16) for amplicon as depicted in SEQ ID NO:12. Expression was normalized to the averaged expression of four housekeeping genes PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), GAPDH (GenBank Accession No. BC026907; amplicon—SEQ ID NO:27; forward primer—SEQ ID NO:28; reverse primer—SEQ ID NO:29) and SDHA (GenBank Accession No. NM_{—}004168; amplicon—SEQ ID NO:30; forward primer—SEQ ID NO:31; reverse primer—SEQ ID NO:32). Note, the number of samples that showed at least 10 fold over-expression, out of the total number of tested samples, is indicated in FIG. 6a below each cancer subtype.

FIG. 7 is a histogram showing the relative expression of UbcH10 variants (e.g., variants as depicted in SEQ ID NO:1 or 2) in normal and tumor derived lung samples as determined by real time PCR using primers (SEQ ID NOs: 17 and 18) for amplicon as depicted in SEQ ID NO:13. Expression was normalized to the averaged expression of four housekeeping genes PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), Ubiquitin (GenBank Accession No. BC000449; amplicon—SEQ ID NO:33; forward primer—SEQ ID NO:34; reverse primer—SEQ ID NO:35) and SDHA (GenBank Accession No. NM_{—}004168; amplicon—SEQ ID NO:30; forward primer—SEQ ID NO:31; reverse primer—SEQ ID NO:32). Note, the number of samples that showed at least 10 fold over-expression, out of the total number of tested samples, is indicated below each cancer subtype.

FIG. 8 is a histogram showing the relative expression of UbcH10 variants (e.g., transcripts as depicted in SEQ ID NO:1 or 2) in normal, benign and tumor derived ovarian samples as determined by real time PCR using primers (SEQ ID NOs:17 and 18) for amplicon as depicted in SEQ ID NO:13. Expression was normalized to the averaged expression of four housekeeping genes PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), GAPDH (GenBank Accession No. BC026907; amplicon—SEQ ID NO:27; forward primer—SEQ ID NO:28; reverse primer—SEQ ID NO:29) and SDHA (GenBank Accession No. NM_{—}004168; amplicon—SEQ ID NO:30; forward primer—SEQ ID NO:31; reverse primer—SEQ ID NO:32). Note, the number of samples that showed at least 10 fold over-expression, out of the total number of tested samples, is indicated below each cancer subtype.

FIG. 9 is a histogram showing the relative expression of UbcH10 variants (e.g., variants as depicted in SEQ ID NO:1 or 2) in normal and tumor derived lung samples as determined by real time PCR using primers (SEQ ID NOs:19 and 20) for amplicon as depicted in SEQ ID NO:14. Expression was normalized to the averaged expression of four housekeeping genes PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), Ubiquitin (GenBank Accession No. BC000449; amplicon—SEQ ID NO:33; forward primer—SEQ ID NO:34; reverse primer—SEQ ID NO:35) and SDHA (GenBank Accession No. NM_{—}004168; amplicon—SEQ ID NO:30; forward primer—SEQ ID NO:31; reverse primer—SEQ ID NO:32). Note, the number of samples that showed at least 10 fold over-expression, out of the total number of tested samples, is indicated below each cancer subtype.

FIG. 10 is a histogram showing the relative expression of UbcH10 variants (e.g., variants as depicted in SEQ ID NO:1 or 2) in normal, benign and tumor derived ovarian samples as determined by real time PCR using primers (SEQ ID NOs:19 and 20) for amplicon as depicted in SEQ ID NO:14. Expression was normalized to the averaged expression of four housekeeping genes PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), GAPDH (GenBank Accession No. BC026907; amplicon—SEQ ID NO:27; forward primer—SEQ ID NO:28; reverse primer—SEQ ID NO:29) and SDHA (GenBank Accession No. NM_{—}004168; amplicon—SEQ ID NO:30; forward primer—SEQ ID NO:31; reverse primer—SEQ ID NO:32). Note, the number of samples that showed at least 10 fold over-expression, out of the total number of tested samples, is indicated below each cancer subtype.

FIGS. 11a-c depict the alignment of the WT UbcH10 (000762; SEQ ID NO:11) protein to the UbcH10 Variants of the present invention (SEQ ID NOs:4, 5 and 6). The alignment was created using Blast P 2.2.3 (Apr. 24, 2002), (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402). FIG. 11a is an alignment of the UbcH10 Variant of SEQ ID NO:4. FIG. 11b is an alignment of the UbcH10 Variant of SEQ ID NO:5. FIG. 11c is an alignment of the UbcH10 Variant of SEQ ID NO:6. The sequence of WT UbcH10 (SEQ ID NO:11) is shown in black and the sequences of each of the UbcH10 variants (e.g., SEQ ID NOs:4, 5, and 6) are shown in red.

FIG. 12 is a schematic summary of quantitative real-time PCR analysis.

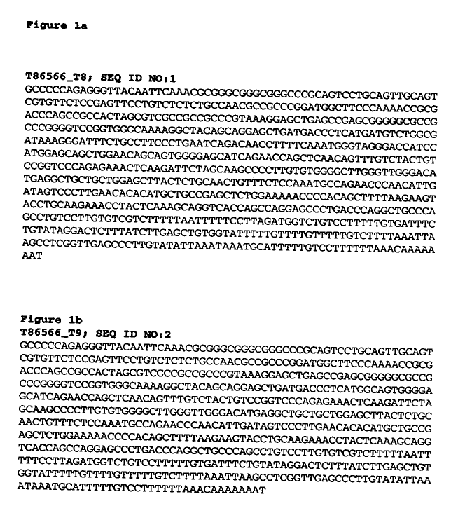

### DESCRIPTION OF THE PREFERRED EMBODIMENTS

The present invention is of novel UbcH10 transcripts and polypeptides which can be used in diagnosis, prognosis, prediction, screening, early diagnosis, staging, therapy selection, treatment and treatment monitoring of UbcH10-related diseases, such as cancer.

As used herein the phrase “a UbcH10 polypeptide” refers to at least an active portion (as is further described hereinbelow) of a naturally occurring protein product of a UbcH10 gene and homologues thereof (GenBank Accession Nos: 000762 and UBCC_HUMAN).

As used herein the phrase “UbcH10-related disease” refers to a disease which is dependent on normal or abnormal expression or activity of a UbcH10 polypeptide for its onset and/or progression; and/or is associated with abnormal activity or expression of a UbcH10 biomolecular sequence.

Examples of UbcH10-related disease types include, but are not limited to cancer such as bladder cancer, breast cancer, testis cancer, cancers of the central nervous system (e.g., head and neck), sarcomas, prostate cancer, pancreatic cancer, ovarian cancer, lung cancer, gastric cancer, esophageal cancer, endometrial cancer, colorectal cancer, salivary gland cancer, renal cancer, oral cancer and cervical cancer; neuronal diseases such as akathesia, Alzheimer's disease, amnesia, amyotrophic lateral sclerosis, bipolar disorder, catatonia, cerebral neoplasms, dementia, depression, diabetic neuropathy, Down's syndrome, tardive dyskinesia, dystonias, epilepsy, Huntington's disease, peripheral neuropathy, multiple sclerosis, neurofibromatosis, Parkinson's disease, paranoid psychoses, postherpetic neuralgia, schizophrenia, and Tourette's disorder; and autoimmune disorders such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Wemer syndrome, complications of cancer, hemodialysis, and extracorporeal circulation, viral, bacterial, fungal, parasitic, protozoal, and helminthic infections, and trauma (U.S. Pat. No. 6,277,568).

According to preferred embodiments of this aspect of the present invention a UbcH10-related disease is lung cancer or ovarian cancer.

As used herein the phrase “UbcH10 related cancer(s)” refers to cancers, where UbcH10 transcripts are differentially expressed as compared to non-cancerous conditions. UbcH10 related cancers include, but are not limited to, lung cancer and ovarian cancer.

In another embodiment, the present invention relates to bridges, tails, heads and/or insertions, and/or analogs, homologs and derivatives of such peptides. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding “known protein”, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding “known protein”.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

As used herein the phrase “known protein” refers to a wild type or other database provided sequence of a specific protein, i.e., any amino acid sequence of a protein which is available in any database as of Jan. 13, 2004, including, but not limited to, SwissProt (http://ca.expasy.org/), National Center of Biotechnology Information (NCBI )(http://www.ncbi.nlm.nih.gov/), PIR (http://pir.georgetown.edu/), A Database of Human Unidentified Gene-Encoded Large Proteins [HUGE <http://www.kazusa.orjp/huge>], Nuclear Protein Database [NPDhttp://npd.hgu.mrc.ac.uk], human mitochondrial protein database (http://bioinfo.nist.gov:8080/examples/servlets/index.html), and University Protein Resource (UniProt) (http://www.expasy.uniprot.org/).

As used herein, the term “homologous” when used to relate amino acid sequences reflects a level of identity and similarity between the sequences.

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

According to still other preferred embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Diagnostics

The term “marker” in the context of the present invention refers to a nucleic acid fragment, a peptide, or a polypeptide, which is differentially present in a sample taken from patients having UbcH10 related cancer as compared to a comparable sample taken from subjects who do not have a UbcH10 related cancer.

The methods for detecting these markers have many applications. For example, one marker or combination of markers can be measured to differentiate between various types of UbcH10 related cancers, and thus are useful as an aid in the accurate diagnosis of UbcH10 related cancers in a patient. For example, one marker or combination of markers can be measured to differentiate between various types of lung cancers, such as small cell or non-small cell lung cancer, and further between non-small cell lung cancer types, such as adenocarcinomas, squamous cell and large cell carcinomas, and thus are useful as an aid in the accurate diagnosis of lung cancer in a patient. In another example, the present methods for detecting these markers can be applied to in vitro UbcH10 related cancers cells or in vivo animal models for UbcH10 related cancers to assay for and identify compounds that modulate expression of these markers.

The phrase “differentially present” refers to differences in the quantity of a marker present in a sample taken from patients having UbcH10 related cancer as compared to a comparable sample taken from patients who do not have UbcH10 related cancer. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. One of ordinary skill in the art could easily determine such relative levels of the markers; further guidance is provided below.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The phrase “predisposition” used herein refers to the susceptibility to develop a disorder. A subject with a predisposition to develop a disorder is more likely to develop the disorder than a non-predisposed subject.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease.

As used herein “a biological sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, sputum, milk, blood cells, tumors, neuronal tissue, organs, and also samples of in vivo cell culture constituents. It should be noted that a “biological sample obtained from the subject” may also optionally comprise a sample that has not been physically removed from the subject, as described in greater detail below.

As used herein, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual.

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of a UbcH10 related cancer or other UbcH10 related disease. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a patient with UbcH10 related cancer or other UbcH10 related disease or a person without cardiac disease. A control amount can be either in absolute amount (e.g., microgram/ml) or a relative amount (e.g., relative intensity of signals).

“Detect” refers to identifying the presence, absence or amount of the object to be detected.

A “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavidin. The label may be directly or indirectly detectable. Indirect detection can involve the binding of a second label to the first label, directly or indirectly. For example, the label can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with” when referring to a protein or peptide (or other epitope), refers to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides a method for detecting a splice variant according to the present invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting the interaction; wherein the presence of an interaction correlates with the presence of a splice variant in the biological sample.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

According to another embodiment of the present invention the detection of the splice variant nucleic acid sequences in the biological sample is effected by detecting at least one nucleic acid change within a nucleic acid material derived from the biological sample; wherein the presence of the at least one nucleic acid change correlates with the presence of a splice variant nucleic acid sequence in the biological sample.

According to the present invention, the splice variants described herein are non-limiting examples of markers for diagnosing UbcH10 related cancer or other UbcH10 related disease and/or pathology. Each splice variant marker of the present invention can be used alone or in combination, for various uses, including but not limited to, prognosis, prediction, screening, early diagnosis, determination of progression, therapy selection and treatment monitoring of such a cancer, disease or pathology.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

According to other preferred embodiments of the present invention, a splice variant protein or a fragment thereof, or a splice variant nucleic acid sequence or a fragment thereof, may be featured as a biomarker for detecting UbcH10 related cancer, disease or pathology, such that a biomarker may optionally comprise any of the above.

Non-limiting examples of methods or assays are described below.

The present invention also relates to kits based upon such diagnostic methods or assays.

Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). U.S. patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No: 6,303,374.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No: 6,303,374.

It is not necessary for all positions in a given oligonucleotide molecule to be uniformly modified, and in fact more than one of the aforementioned modifications may be incorporated in a single compound or even at a single nucleoside within an oligonucleotide.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No: 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byme et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (±), pGL3, PzeoSV2 (±), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., includingRetro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, RNA in-situ hybridization, in-situ RT-PCR, primer extension, Southern blots (DNA detection), dot or slot blots (DNA, RNA), Northern blots (RNA detection) and other NAT type assays which are further described in greater detail below. More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACl, 0.01 M sodium phosphate (pH 6.8), 1 mM EDTA (pH7.6), 0.5% SDS, 100 μg/ml denatured salmon sperm DNA and 0.1% nonfat dried milk, hybridization temperature of 1-1.5° C. below the Tm, final wash solution of 3 M TMACl, 0.01 M sodium phosphate (pH6.8), 1 mM EDTA (pH7.6), 0.5% SDS at 1-1.5° C. below the Tm; (ii) hybridization solution of 6×SSC and 0.1% SDS or 3 M TMACl, 0.01 M sodium phosphate (pH6.8), 1 mM EDTA (pH7.6), 0.5% SDS, 100 μg/ml denatured salmon sperm DNA and 0.1% nonfat dried milk, hybridization temperature of 2-2.5° C. below the Tm, final wash solution of 3 M TMACl, 0.01 M sodium phosphate (pH6.8), 1 mM EDTA (pH7.6), 0.5% SDS at 1-1.5° C. below the Tm, final wash solution of 6×SSC, and final wash at 22° C.; (iii) hybridization solution of 6×SSC and 1% SDS or 3 M TMACl, 0.01 M sodium phosphate (pH6.8), 1 mM EDTA (pH7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well-known methods. Non-limiting examples of radioactive labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well-known methods.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

Preferably, the present invention can also utilize PNA probes for detecting the splice variant sequences of the present invention. PNA probes are synthetic DNA mimics in which the sugar phosphate backbone is replaced by repeating N-(2-aminoethyl) glycine units linked by an amine bond and to which the nucleobases are fixed (Pellestor F and Paulasova P, 2004; Chromosoma 112: 375-380). Thus, the hydrophobic and neutral backbone enables high affinity and specific hybridization of the PNA probes to their nucleic acid counterparts (e.g., chromosomal DNA or genomic DNA).

As is mentioned before, hybridization according to the present invention can be also effected on a biological sample containing RNA molecules using methods such as Northern Blot analysis and RNA in situ hybridization stain.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

NAT Assays

Detection of a nucleic acid of interest in a biological sample may also optionally be effected by NAT-based assays, which involve nucleic acid amplification technology, such as PCR, or variations thereof (e.g., real-time PCR, RT-PCR and in situ RT-PCR).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14. Numerous amplification techniques have been described and can be readily adapted to suit particular needs of a person of ordinary skill. Non-limiting examples of amplification.-techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are the to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo-and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)n=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 220, or 1,048,576 copies of the starting material. If the reaction conditions reduce the mean efficiency to 85%, then the yield in those 20 cycles will be only 1.8520, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The NAT assays of the present invention also include methods of detecting at least one nucleic acid change [e.g., a single nucleotide polymorphism (SNP] in the biological sample of the present invention.

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations or nucleic acid changes. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G−C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses Acyclopol™, a novel mutant thermostable polymerase from the Archeon family, and a pair of AcycloTerminators™ labeled with the fluorescent dyes R110 and TAMRA, representing the possible alleles for the SNP of interest. AcycloTerminator™ non-nucleotide analogs are biologically active with a variety of DNA polymerases. Similarly to 2′, 3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutants have for derivatized 2′, 3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

It will be appreciated that advances in the field of SNP detection have provided additional accurate, easy, and inexpensive large-scale SNP genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], , the TaqMan system (Holland, P. M. et al., 1991. Detection of specific polymerase chain reaction product by utilizing the 5′-3′ exonuclease activity of Thermus aquaticus DNA polymerase. Proc Natl Acad Sci U S A. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S. and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol Diagn. 5: 341-80).

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis, dideoxy fingerprinting, Pyrosequencing™, Acycloprime™, and reverse dot blot.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

Preferably, the detection of at least one nucleic acid change and/or the splice variant sequence of the present invention is effected in a biological sample containing RNA molecules using, for example, RT-PCR or in situ RT-PCR.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

It will be appreciated that when utilized along with automated equipment, the above described detection methods can be used to screen multiple samples for a disease and/or pathological condition both rapidly and easily.

Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C-NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| isoleucine | Iie | I |
| Leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as | Xaa | X |
| above | | |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Antibodies

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Preferably, the antibody of this aspect of the present invention specifically binds at least one epitope of the polypeptide variants of the present invention.

As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA). In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Light Emission Immunoassay: This method is based on covalently attaching a substrate (e.g., firefly luciferin) to specific antibodies and following the emission of light of the bound antibody on the tested cells or tissue (see for example, Schaeffer J M, Hsueh A J., 1984; J. Biol. Chem. 259: 2055-8).

Radio-imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12;274(4):622-34; Giebel L B et al. “Screening of cyclic peptide phage libraries identifies ligands that bind streptavidin with high affinities” Biochemistry 1995 Nov. 28;34(47):15430-5; Davies E L et al., “Selection of specific phage-display antibodies using libraries derived from chicken immunoglobulin genes” J Immunol Methods 1995 Oct. 12;186(l):125-35; Jones C R T al. “Current trends in molecular recognition and bioseparation” J Chromatogr A 1995 Jul. 14;707(l):3-22; Deng S J et al. “Basis for selection of improved carbohydrate-binding single-chain antibodies from synthetic gene libraries” Proc Natl Acad Sci U S A 1995 May 23;92(11):4992-6; and Deng S J et al. “Selection of antibody single-chain variable fragments with improved carbohydrate binding by phage display” J Biol Chem 1994 Apr. 1;269(13):9533-8, which are incorporated herein by reference.

Treatment

As mentioned hereinabove the UbcH10 variants of the present invention and compositions derived therefrom (i.e., peptides, oligonucleotides) can be used to treat a subject having, being diagnosed with or predisposed to a UbcH10-related disease, such as cancer.

The subject according to the present invention is a mammal, preferably a human which is diagnosed with one of the diseases described hereinabove, or alternatively is predisposed to having one of the diseases described hereinabove.

As used herein the term “treating” refers to preventing, curing, reversing, attenuating, alleviating, minimizing, suppressing or halting the deleterious effects of the UbcH10-related disease.

Treating according to the present invention is effected by specifically upregulating the expression in the subject of at least one of the polypeptides of the present invention. As described hereinabove upregulation of the polypeptides of the present invention or active portions thereof can result in for example cell-cycle arrest, which is desired in hyperproliferative diseases (i.e., cancer).

As used hereinabove the phrase “active portion” refers to an amino acid sequence portion which is capable of displaying one or more functions of the UbcH10 polypeptides of the present invention. Examples include but are not limited to E1 binding, E3 binding, cell cycle arrest and antibody specific recognition.

Upregulating Methods and Agents

Upregulating expression of the UbcH10 variants of the present invention may be effected via the administration of at least one of the exogenous polynucleotide sequences of the present invention (e.g., SEQ ID NOs:1-3, 9-10 and/or 12-14) ligated into a nucleic acid expression construct designed for expression of coding sequences in eukaryotic cells (e.g., mammalian cells). Accordingly, the exogenous polynucleotide sequence may be a DNA or RNA sequence encoding the variants of the present invention or active portions thereof.

It will be appreciated that the nucleic acid construct can be administered to the individual employing any suitable mode of administration, described hereinbelow (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

To enable cellular expression of the polynucleotides of the present invention, the nucleic acid construct of the present invention further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. Coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (±), pGL3, PzeoSV2 (±), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

It will be appreciated that the present methodology may also be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-SR gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Molecular and Subcellular Biology 31:217-239.

Alternatively or additionally, upregulation may be effected by administering to the subject at least one polypeptide agent of the polypeptides of the present invention or an active portion thereof, as described hereinabove. However, since the bioavailability of large polypeptides is relatively small due to high degradation rate and low penetration rate, administration of polypeptides is preferably confined to small peptide fragments (e.g., about 100 amino acids).

An agent capable of upregulating a UbcH10 polypeptide may also be any compound which is capable of increasing the transcription and/or translation of an endogenous DNA or mRNA encoding the UbcH10 polypeptide and thus increasing endogenous UbcH10 activity.

An agent capable of upregulating a UbcH10 may also be an exogenous polypeptide including at least a functional portion (as described hereinabove) of the UbcH10.

Upregulation of UbcH10 can be also achieved by introducing at least one UbcH10 substrate. Non-limiting examples of such agents include HOXC10 (Gabellini D, et al., 2003; EMBO J. 22: 3715-24), human securin and cyclin B1 (Tang Z, et al., 2001; Mol. Biol. Cell. 12: 3839-51), cyclins A, geminin H, and Cut2p (Bastians H, et al., 1999; Mol. Biol. Cell. 10: 3927-3941).

It will be appreciated that upregulation of UbcH10 can be also effected by administration of UbcH10-expressing cells into the individual.

UbcH10-expressing cells can be any suitable cells, such as lung, ovary, bone marrow which are derived from the individual and are transfected ex vivo with an expression vector containing the polynucleotide designed to express UbcH10 as described hereinabove.

Administration of the UbcH10-expressing cells of the present invention can be effected using any suitable route such as intravenous, intra peritoneal, and intra ovary. According to presently preferred embodiments, the UbcH10-expressing cells of the present invention are introduced to the individual using intravenous and/or intra organ administrations.

UbcH10-expressing cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002;13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Downregulating Methods and Agents

Downregulation of UbcH10 can be effected on the genomic and/or the transcript level using a variety of molecules which interfere with transcription and/or translation (e.g., antisense, siRNA, Ribozyme, DNAzyme), or on the protein level using e.g., antagonists, enzymes that cleave the polypeptide and the like.

Following is a list of agents capable of downregulating expression level and/or activity of UbcH10.

One example, of an agent capable of downregulating a UbcH10 polypeptide is an antibody or antibody fragment capable of specifically binding UbcH10. Preferably, the antibody specifically binds at least one epitope of a UbcH10 as described hereinabove.

An agent capable of downregulating a UbcH10 transcript is a small interfering RNA (siRNA) molecule. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the UbcH10 transcript mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl, T. 2001, ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating a UbcH10 transcript is a DNAzyme molecule capable of specifically cleaving an mRNA transcript or DNA sequence of the UbcH10. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther. www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a UbcH10 transcript can also be effected by using an antisense polynucleotide capable of specifically hybridizing with an mRNA transcript encoding the UbcH10.

Design of antisense molecules which can be used to efficiently downregulate a UbcH10 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a UbcH10 transcript is a ribozyme molecule capable of specifically cleaving an mRNA transcript encoding a UbcH10. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase I trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Another agent capable of downregulating UbcH10 would be any molecule which binds to and/or cleaves UbcH10. Such molecules can be UbcH10 antagonists, or UbcH10 inhibitory peptide.

It will be appreciated that a non-functional analogue of at least a catalytic or binding portion of UbcH10 can be also used as an agent which downregulates UbcH10.

Another agent which can be used along with the present invention to downregulate UbcH10 is a molecule which prevents UbcH10 activation or substrate binding.

Each of the upregulating or downregulating agents described hereinabove or the expression vector encoding UbcH10 can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that treatment of UbcH10 related disease according to the present invention may be combined with other treatment methods known in the art (i.e., combination therapy). Thus, treatment of UbcH10-related cancer may be combined with, for example, radiation therapy, antibody therapy and/or chemotherapy.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

In spite of improved treatments for certain forms of cancer, it is still a leading cause of death. Since the chance for complete remission of cancer is, in most cases, greatly enhanced by early diagnosis, it is very desirable to detect cancers before a substantial tumor develops. However, the development of methods that permit rapid and accurate detection and treatment of many forms of cancers continues to challenge the medical community.

While reducing the present invention to practice, the present inventors have uncovered novel variants of UbcH10, which are over expressed in various types of cancer. These findings provide overwhelming evidence that the newly discovered variants of UbcH10 can be utilized in both diagnosis and treatment of cancer.

These markers are overexpressed in UbcH10 related cancers specifically, as opposed to non-cancerous tissues. The measurement of these markers, alone or in combination, in patient samples provides information that the diagnostician can correlate with a probable diagnosis of UbcH10 related cancers. The markers of the present invention, alone or in combination, show a high degree of differential detection between UbcH10 related cancers and non-cancerous states. The markers of the present invention, alone or in combination, can be used for prognosis, prediction, screening, early diagnosis, staging, therapy selection and treatment monitoring of UbcH10 related cancers. For example, optionally and preferably, these markers may be used for staging UbcH10 related cancers and/or monitoring the progression of the disease. Furthermore, the markers of the present invention, alone or in combination, can be used for detection of the source of metastasis found in anatomical places other than these where the primary cancer was originally found. Also, one or more of the markers may optionally be used in combination with one or more other cancer markers (other than those described herein).

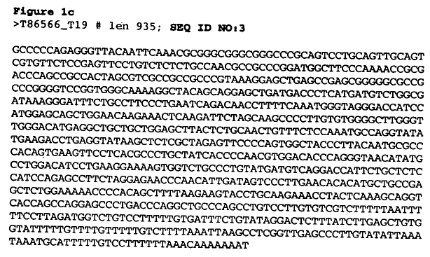

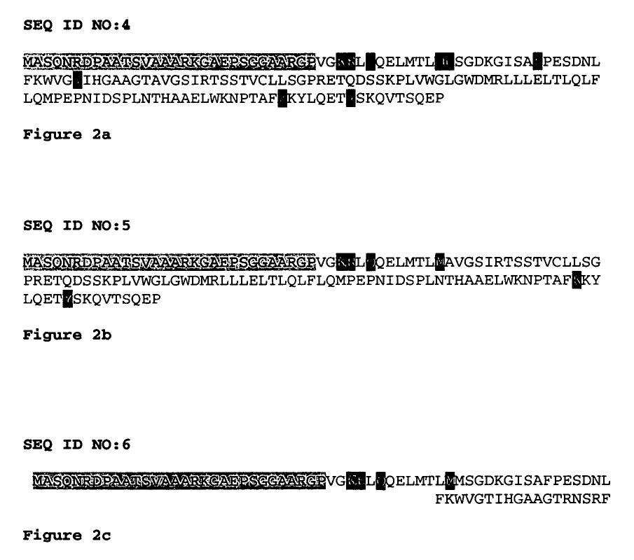

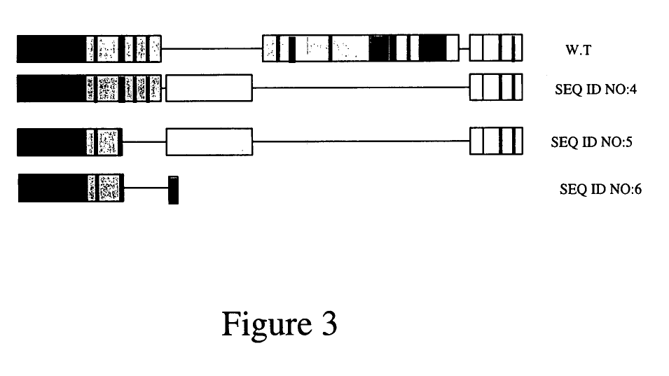

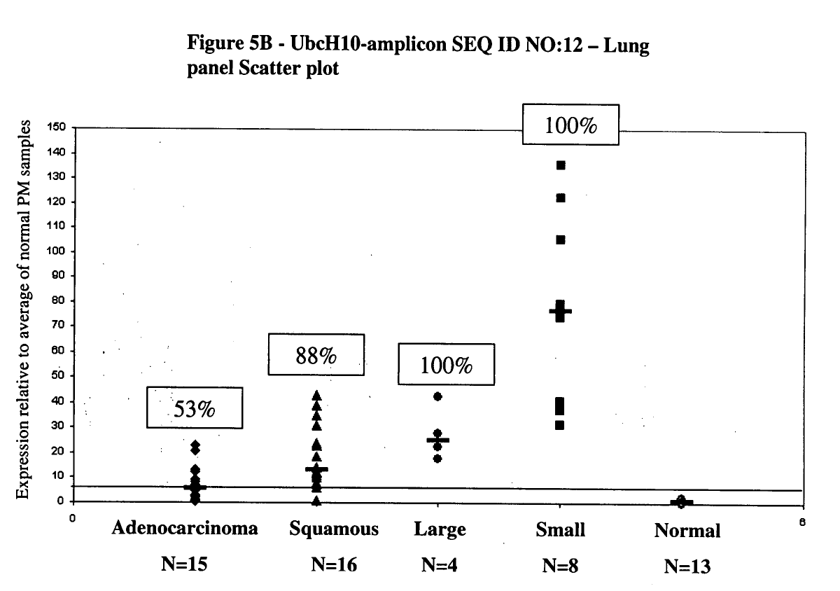

As is further illustrated hereinunder and in the Examples section which follows, the UbcH10 variants uncovered by the present study are splice variants of this ubiquitin conjugating enzyme [Transcript: GenBank Accession No. U73379 (SEQ ID NO:36); Protein: GenBank Accession No. 000762 (SEQ ID NO:11)]. Newly uncovered transcripts SEQ ID NOs:1 and 2 code for novel UbcH10 polypeptides that include a unique amino acid sequence of 50 amino acids (SEQ ID NO:7) (see, yellow box FIGS. 2, 3), while lacking the conserved E2 catalytic site, which includes the catalytic cysteine, which mediates ubiquitin-thiolester formation. Real time PCR analyses showed that UbcH10 variants as depicted in SEQ ID NOs:1 and 2 are over expressed in ovarian and lung cancer tissues (see FIGS. 5-10).

Thus, according to one aspect of the present invention there is provided an isolated polynucleotide comprising a nucleic acid sequence encoding a UbcH10 polypeptide having at least a portion of an amino acid sequence at least 55% homologous to SEQ ID NO:7, as determined using the BlastP software of the National Center of Biotechnology Information (NCBI) using default parameters.

According to one embodiment of this aspect of the present invention the nucleic acid sequence is as set forth in SEQ ID NO:1-3, 9, 10, 12-14.

According to another embodiment of the present invention there is a unique nucleic acid segment, as depicted in SEQ ID NO:9, which is part of the UbcH10 variant sequences as set forth in SEQ ID NOs:1 or 2.

According to another embodiment of the present invention there is a unique nucleic acid segment, as depicted in SEQ ID NO:10, which is part of the UbcH10 variant sequences as set forth in SEQ ID NOs:1, 2 or 3.

Preferably, the polynucleotide according to this aspect of the present invention encodes a polypeptide, which is as set forth in SEQ ID NO:4, 5 or 6.

According to a preferred embodiment of this aspect of the present invention the active portion of the polypeptide is as set forth in SEQ ID NO:7 or 8.

According to another aspect of the present invention there is provided an isolated polynucleotide including a nucleic acid sequence at least 60% identical to SEQ ID NO:1, 2 or 3, as determined using the BlastN software of the National Center of Biotechnology Information (NCBI) using default parameters.

Preferably, the isolated polynucleotide includes a nucleic acid sequence at least 60%, least 61%, least 62%, least 63%, least 64%, at least 65%, least 66%, least 67%, least 68%, least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more say 100% identical to SEQ ID NO:1, 2 or 3, as determined using the BlastN software of the National Center of Biotechnology Information (NCBI) using default parameters, which preferably include using the DUST filter program, and also preferably include having an E value of 10, filtering low complexity sequences and a word size of 11.

According to a preferred embodiment of this aspect of the present invention the nucleic acid sequence is as set forth in SEQ ID NO:1, 2 or 3.

Preferably, the polynucleotide according to this aspect of the present invention encodes a UbcH10 polypeptide, which is as at least 50%, at least 55%, at least 60%, at least 65%, at least 70%, at least 75%, at least 80%, %, at least 85%, at least 90%, at least 95% or more say 100% homologous, to the polypeptide set forth in SEQ ID NO:4, 5 or 6, as determined using the BlastP software of the National Center of Biotechnology Information (NCBI) using default parameters.

The isolated polynucleotides of this aspect of the present invention can be qualified using an hybridization assay by incubating the isolated polynucleotides described above with a probe having the sequence set forth in SEQ ID NO:1-3, 9, 10, 12-14 under moderate to stringent hybridization conditions as described hereinabove.

According to preferred embodiments of the present invention the isolated polynucleotides of the present invention can be also qualified using a NAT-based assay as described hereinabove, using primers such as those set forth by SEQ ID NOs:15, 16, 17, 18, 19 and 20.

Thus, the present invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Since the polynucleotide sequences of the present invention encode previously unidentified polypeptides, the present invention also encompasses novel polypeptides of UbcH10 or portions thereof, which are encoded by the isolated polynucleotide and respective nucleic acid fragments thereof described hereinabove.

Thus, the present invention also encompasses polypeptides encoded by the novel UbcH10 variants of the present invention. The amino acid sequences of these novel polypeptides are set forth in SEQ ID NO:4-8. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to SEQ ID NO:4-8. Finally, the present invention also encompasses fragments of the above described polypeptides and polypeptides having mutations, such as deletions, insertions or substitutions of one or more amino acids, either naturally occurring or man induced, either randomly or in a targeted fashion.

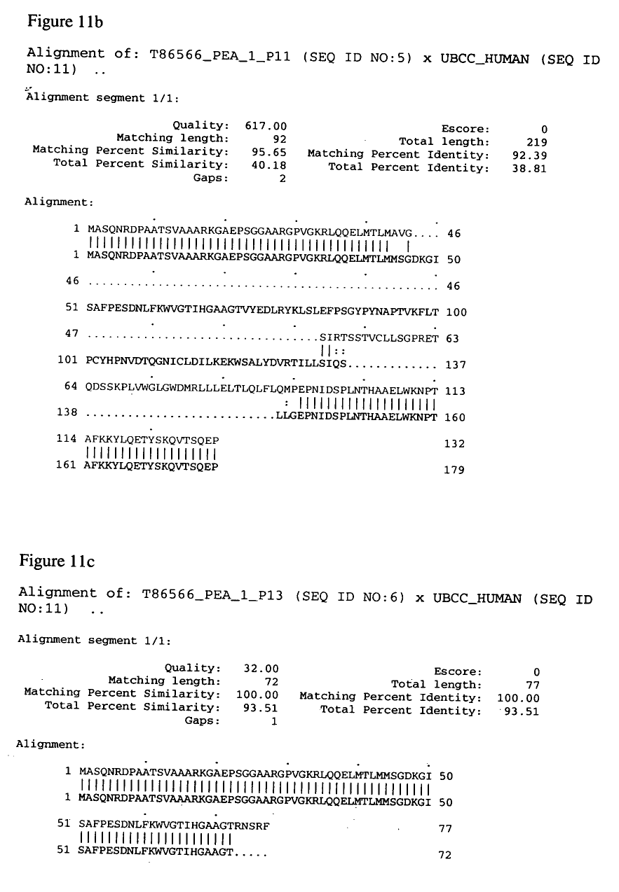

For example, the comparison of the polypeptides, encoded by the novel UbcH10 variants of the present invention, to the WT UbcH10 (SEQ ID NO:11) is demonstrated in FIG. 11. FIG. 11 demonstrates an alignment of the WT UbcH10 (O00762) protein to the UbcH10 Variants of the present invention (SEQ ID NOs:4, 5, 6), using Blast P 2.2.3 (Apr. 24, 2002), (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

According to still a further aspect of the present invention there is provided an isolated polypeptide encoding for UbcH10, comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the edge polypeptide, and wherein the first amino acid, the edge polypeptide and the second amino acid sequence are in a sequential order.

According to preferred embodiments of the present invention, the first amino acid sequence according to this aspect of the present invention is at least 90%, at least 91%, at least 92%, at least 93%, at least 94%, at least 95%, at least 96%, at least 97%, at least 98%, at least 99% homologous to amino acids 1-72 as set forth in SEQ ID NO:11.

Preferably, the first amino acid sequence is according to this aspect of the present invention is set forth by amino acids 1-72 of SEQ ID NO:11.

According to preferred embodiments of the present invention, the edge polypeptide according to this aspect of the present invention has an amino acid sequence at least 70%, least 71%, least 72%, least 73%, least 74%, least 75%, least 76%, least 77%, least 78%, least 79%, least 80%, least 81%, at least 82%, least 83%, least 84%, least 85%, least 86%, least 87%, least 88%, least 89%, least 90%, least 91%, least 92%, least 93%, at least 94%, least 95%, least 96%, least 97%, least 98%, least 99% homologous to the amino acid sequence set forth by SEQ ID NO:7 (AVGSIRTSSTVCLLSGPRETQDSSKPLVWGLGWD MRLLLELTLQLFLQMP).

Preferably, the edge polypeptide according to this aspect of the present invention is set forth by SEQ ID NO:7.

According to preferred embodiments of the present invention the second amino acid sequence according to this aspect of the present invention is at least 90%, at least 91%, at least 92%, at least 93%, at least 94%, at least 95%, at least 96%, at least 97%, at least 98%, at least 99% homologous to amino acids 141-179 of WT UbcH10 (SEQ ID NO:11).

Preferably, the second amino acid sequence according to this aspect of the present invention is set forth by amino acids 141-179 of SEQ ID NO:11.

Preferably, the isolated polypeptide encoding for UbcH10 according to this aspect of the present invention is set forth by SEQ ID NO:4.

According to preferred embodiments of the present invention the edge polypeptide according to this aspect of the present invention includes at least one bridge portion. Preferably, such a polypeptide includes a first bridge portion and a second bridge portion.

According to preferred embodiments the first bridge portion of UbcH10 splice variant according to this aspect of the present invention includes a polypeptide having “n” amino acids, wherein “n” is at least 10, optionally at least about 20, preferably at least about 30, more preferably at least about 40 and most preferably at least about 50, and whereas at least two amino acids of the first bridge portion are Threonine and Alanine, and wherein the first bridge portion has a structure as follows (numbering according to SEQ ID NO:4): a sequence starting from any of amino acid numbers 72−x to 72; and ending at any of amino acid numbers 73+((n−2)−x), in which x varies from 0 to n−2.

For example, for peptides of 10 amino acids (such that n=10), the starting position could be as “early” in the sequence as amino acid number 64 if x=n−2=8 (i.e., 64=72−8), such that the peptide would end at amino acid number 73 (73+(8−8=0)). On the other hand, the peptide could start at amino acid number 72 if x=0 (i.e., 72=72−0), and could end at amino acid 81 (73+(8−0=8)).

The at least one bridge portion above, comprising a polypeptide being at least 70%, optionally at least about 80%, preferably at least about 85%, more preferably at least about 90% and most preferably at least about 95% homologous to at least one sequence described above.

Similarly, the at least one bridge portion according to this aspect of the present invention may optionally be relatively short, such as from about 4 to about 9 amino acids in length. For four amino acids, the first bridge portion would comprise the following peptides: TAVG, GTAV, AGTA. All peptides feature TA as a portion thereof. Peptides of from about five to about nine amino acids could optionally be similarly constructed.

According to preferred embodiments, the second bridge portion according to this aspect of the present invention includes a polypeptide having “n” amino acids, wherein the value of “n” is at least 10, optionally at least 20, preferably at least 30, more preferably at least 40 and most preferably at least 50, and whereas at least two amino acids of the second bridge portion are Proline and Glutamic acid, and wherein the second bridge portion has a structure as follows (numbering according to SEQ ID NO:4): a sequence starting from any of amino acid numbers 122−x to 122; and ending at any of amino acid numbers 123+((n−2)−x), in which x varies from 0 to n−2.

For example, for peptides of 10 amino acids (such that n=10), the starting position could be as “early” in the sequence as amino acid number 114 if x=n−2=8 (i.e., 114=122−8), such that the peptide would end at amino acid number 123 (123+(8−8=0)). On the other hand, the peptide could start at amino acid number 122 if x=0 (i.e., 122=122−0), and could end at amino acid 131 (123+(8−0=8)).

The second bridge portion above, comprising a polypeptide being at least 70%, optionally at least about 80%, preferably at least about 85%, more preferably at least about 90% and most preferably at least about 95% homologous to at least one sequence described above.

Similarly, the second bridge portion may optionally be relatively short, such as from about 4 to about 9 amino acids in length. For four amino acids, the second bridge portion would comprise the following peptides: PEPN, MPEP, QMPE. All peptides feature PE as a portion thereof. Peptides of from about five to about nine amino acids could optionally be similarly constructed.

According to another aspect of the present invention there is provided an isolated polypeptide encoding for UbcH10 new variant including a first amino acid sequence being at least 90% homologous to amino acids 1-43 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:11, wherein the first amino acid is contiguous to the edge polypeptide and the second amino acid sequence is contiguous to the bridge polypeptide, and wherein the first amino acid, the edge polypeptide and the second amino acid sequence are in a sequential order.

According to preferred embodiments of the present invention the first amino acid sequence is at least 90%, at least 91%, at least 92%, at least 93%, at least 94%, at least 95%, at least 96%, at least 97%, at least 98%, at least 99% homologous to amino acids 1-43 of SEQ ID NO:11 (WT UbcH10, GenBank Accession No. O00762).

Preferably, the first amino acid sequence of the present invention is set forth by amino acids 1-43 of SEQ ID NO:11 or 5.

According to preferred embodiments of the present invention, the edge polypeptide according to this aspect of the present invention has an amino acid sequence at least 70%, least 71%, least 72%, least 73%, least 74%, least 75%, least 76%, least 77%, least 78%, least 79%, least 80%, least 81%, at least 82%, least 83%, least 84%, least 85%, least 86%, least 87%, least 88%, least 89%, least 90%, least 91%, least 92%, least 93%, at least 94%, least 95%, least 96%, least 97%, least 98%, least 99% homologous to the amino acid sequence set forth by SEQ ID NO:7.

Preferably, the edge polypeptide according to this aspect of the present invention is set forth by SEQ ID NO:7.

According to preferred embodiments the second amino acid sequence according to this aspect of the present invention is at least about 90%, at least 91%, at least 92%, at least 93%, at least 94%, at least 95%, at least 96%, at least 97%, at least 98%, at least 99% homologous to amino acids 141-179 of WT UbcH10 (SEQ ID NO:11).

Preferably, the second amino acid sequence according to this aspect of the present invention is set forth by amino acids 141-179 of SEQ ID NO:11.

Preferably, the isolated polypeptide encoding for UbcH10 according to this aspect of the present invention is set forth by SEQ ID NO:5.

Preferably, the edge polypeptide according to this aspect of the present invention is set forth by SEQ ID NO:7.

According to preferred embodiments the first bridge portion of the UbcH10 splice variant according to this aspect of the present invention includes a polypeptide having a value of “n” amino acids, wherein “n” is at least 10, optionally at least about 20, preferably at least about 30, more preferably at least about 40 and most preferably at least about 50, and whereas at least two amino acids of the first bridge portion are Methionine and Alanine, and wherein the first bridge portion has a structure as follows (numbering according to SEQ ID NO:5): a sequence starting from any of amino acid numbers 42−x to 42; and ending at any of amino acid numbers 43+((n−2)−x), in which x varies from 0 to n−2.

For example, for peptides of 10 amino acids (such that n=10), the starting position could be as “early” in the sequence as amino acid number 34 if x=n−2=8 (i.e., 34=42−8), such that the peptide would end at amino acid number 43 (43+(8−8=0)). On the other hand, the peptide could start at amino acid number 42 if x=0 (i.e., 42=42−0), and could end at amino acid 51 (43+(8−0=8)).

The at least one bridge portion above, comprising a polypeptide being at least 70%, optionally at least about 80%, preferably at least about 85%, more preferably at least about 90% and most preferably at least about 95% homologous to at least one sequence described above.

Similarly, the at least one bridge portion may optionally be relatively short, such as from about 4 to about 9 amino acids in length. For four amino acids, the first bridge portion would comprise the following peptides: MAVG, LMAV, TLMA. All peptides feature MA as a portion thereof. Peptides of from about five to about nine amino acids could optionally be similarly constructed.

According to preferred embodiments, the second bridge portion according to this aspect of the present invention includes a polypeptide having “n” amino acids, wherein the value of “n is at least 10, optionally at least 20, preferably at least 30, more preferably at least 40 and most preferably at least 50, and whereas at least two amino acids of the second bridge portion are Proline and Glutamic acid, and wherein the second bridge portion has a structure as follows (numbering according to SEQ ID NO:5): a sequence starting from any of amino acid numbers 93−x to 93; and ending at any of amino acid numbers 94+((n−2)−x), in which x varies from 0 to n−2.

For example, for peptides of 10 amino acids (such that n=10), the starting position could be as “early” in the sequence as amino acid number 85 if x=n−2=8 (i.e., 85=93−8), such that the peptide would end at amino acid number 94 (94+(8−8=0)). On the other hand, the peptide could start at amino acid number 93 if x=0 (i.e., 93=93−0), and could end at amino acid 102 (94+(8−0=8)).

The second bridge portion above, comprising a polypeptide being at least 70%, optionally at least about 80%, preferably at least about 85%, more preferably at least about 90% and most preferably at least about 95% homologous to at least one sequence described above.

Similarly, the second bridge portion may optionally be relatively short, such as from about 4 to about 9 amino acids in length. For four amino acids, the second bridge portion would comprise the following peptides: PEPN, MPEP, QMPE. All peptides feature PE as a portion thereof. Peptides of from about five to about nine amino acids could optionally be similarly constructed.

According to yet another aspect of the present invention, there is provided an isolated polypeptide encoding for UbcH10, including a first amino acid sequence being at least 90% homologous to amino acids 1-72 as set forth in SEQ ID NO:11 (WT UbcH10 corresponding to GenBank Accession No. 000762), and a second amino acid sequence being at least 80% homologous to amino acid sequence as set forth of SEQ ID NO:8 (RNSRF; in transcript of SEQ ID NO:3, which corresponds to amino acids 73-77 of SEQ ID NO:6), wherein the first amino acid and the second amino acid sequence are contiguous and in a sequential order.

Preferably, the isolated polypeptide according to this aspect of the present invention is set forth in SEQ ID NO:6.

According to preferred embodiments of the present invention a bridge portion between the first amino acid sequence and the second amino acid sequence according to this aspect of the present invention is a polypeptide having “n” amino acids, wherein “n” is at least 10, optionally at least 20, preferably at least 30, more preferably at least 40, most preferably at least 50, and whereas at least two amino acids of the bridge portion are Threonine and Arginine, and wherein the bridge portion has a structure as follows (numbering according to SEQ ID NO:6): a sequence starting from any of amino acid numbers 72−x to 72; and ending at any of amino acid numbers 73+((n−2)−x), in which x varies from 0 to n−2 such that the value ((n−2)−x) is not allowed to be larger than 4.

For example, for peptides of 10 amino acids (such that n=10), the starting position could be as “early” in the sequence as amino acid number 64 if x=n−2=8 (i.e., 64=72−8), such that the peptide would end at amino acid number 73 (73+(8−8=0)). On the other hand, for ((n−2)−x)=4, the peptide could start at amino acid number 68 (i.e. 68=72−4), and could end at amino acid 77 (73+4).

The at least one bridge portion above, comprising a polypeptide being at least 70%, optionally at least about 80%, preferably at least about 85%, more preferably at least about 90% and most preferably at least about 95% homologous to at least one sequence described above.

Similarly, the at least one bridge portion may optionally be relatively short, such as from about 4 to about 9 amino acids in length. For four amino acids, the first bridge portion would comprise the following peptides: AGTR, GTRN, TRNS. All peptides feature TR as a portion thereof. Peptides of from about five to about nine amino acids could optionally be similarly constructed.

According to another aspect of the present invention there is provided an isolated polypeptide encoding for a tail of UbcH10 new variant as set forth in SEQ ID NO:6, comprising a polypeptide being at least about 80%, preferably at least about 85%, more preferably at least about 90% and most preferably at least about 95% homologous to the sequence RNSRF as set forth by SEQ ID NO:8 (in transcript of SEQ ID NO:3).

A non-limiting example of a unique protein “tail” sequence is the amino acid sequence as set forth in SEQ ID NO:8 (in transcript of SEQ ID NO:3).

According to still an additional aspect of the present invention there is provided an antibody fragment being capable of specifically binding any of the isolated polypeptides of the present invention which are described hereinabove.

According to still an additional aspect of the present invention there is provided an oligonucleotide specifically hybridizable with a nucleic acid sequence encoding a UbcH10 polypeptide including at least a portion of an amino acid sequence at least 55% homologous to SEQ ID NO:7 or 8, as determined using the BlastP software of the National Center of Biotechnology Information (NCBI) using default parameters.

Preferably, the oligonucleotide according to this aspect of the present invention is as set forth in SEQ ID NO:1, 2 or 3.

According to preferred embodiments of the present invention the oligonucleotide is a single or double stranded, and at least 10 bases long. It will be appreciated that such an oligonucleotide is hybridizable in either sense or antisense orientation.

As is mentioned before and is described in Examples 1-6 of the Examples section which follows, the isolated polynucleotides of the present invention (i.e., the new UbcH10 splice variants) were found to be overexpressed in various UbcH10-related cancers (e.g., lung and ovary cancer) and thus can be used in diagnosing and/or determining predisposition to such cancers.

Thus, according to yet an additional aspect of the present invention there is provided a method of diagnosing predisposition to, or presence of UbcH10-related disease in a subject.

The method is effected by determining a level of a UbcH10 polypeptide including at least a portion of an amino acid sequence at least 55% homologous to SEQ ID NO:7 or 8, as determined using the BlastP software of the National Center of Biotechnology Information (NCBI) using default parameters, or of a polynucleotide encoding the polypeptide in a biological sample obtained from the subject, wherein the level of the polynucleotide or the level of the polypeptide is correlatable with predisposition to, or presence or absence of the UbcH10-related disease, thereby diagnosing predisposition to, or presence of UbcH10-related disease in the subject.

As is mentioned hereinabove, UbcH10 polypeptides can be detected in a biological sample using various immunological detection methods as described hereinabove (e.g., RIA, Western Blot, FACS, ELISA, Immunohistochemistry) and with the specific antibody or antibody fragment of the present invention.

Determination of the level of the polynucleotide of the present invention can be effected using hybridization (with DNA or RNA molecules as a template and/or probe) or using NAT-based assays as described hereinabove.

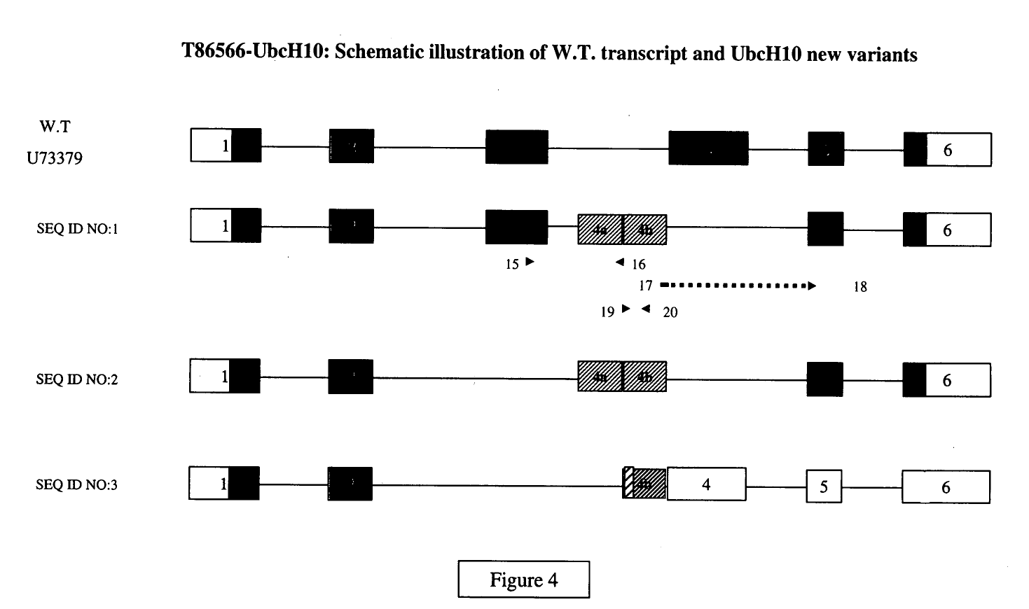

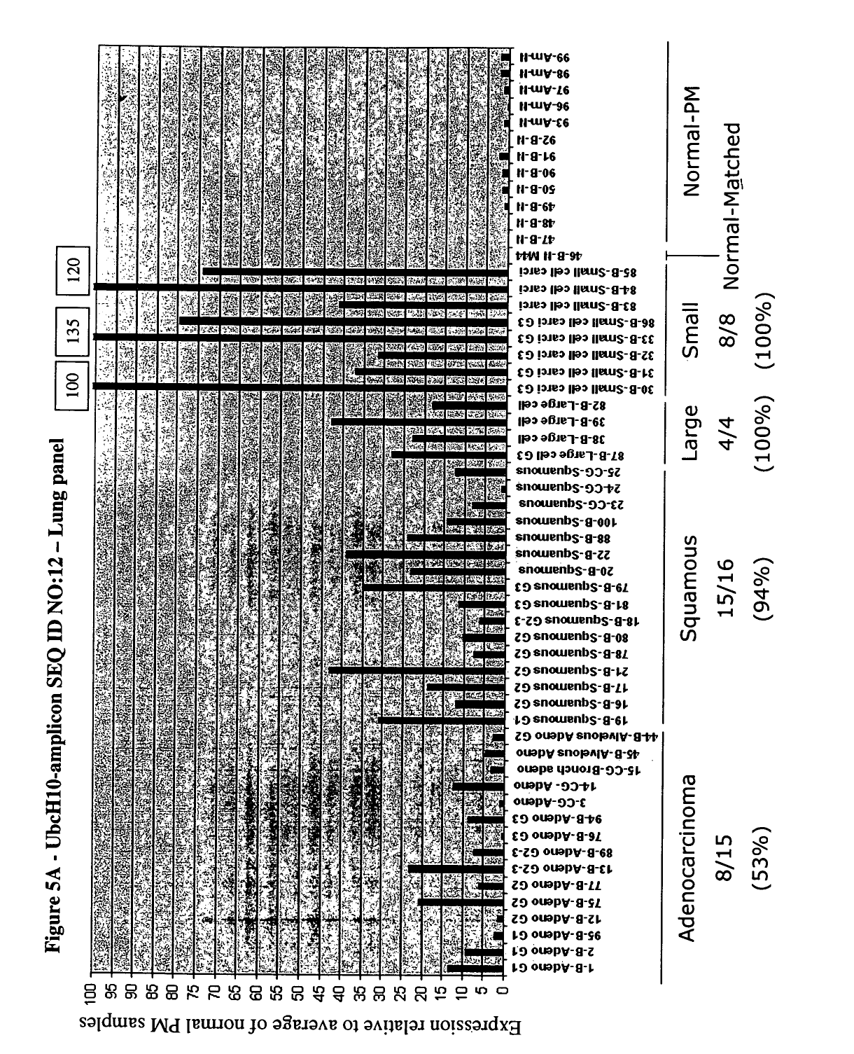

An example of an oligonucleotide probe which can be utilized to detect transcripts SEQ ID NO:1 for example is set forth in SEQ ID NO:12, 13 or 14. An example of an oligonucleotide probe which can be utilized to detect transcripts SEQ ID NOs:2 and 3 for example is set forth in SEQ ID NO:13 or 14. True identification of a single variant such as transcript as set forth in SEQ ID NO:1 is preferably further effected by gel electrophoresis which examines the molecular weight of the variant. Alternatively identification of single variants can be effected using oligonucleotides which are directed to transition sequences bridging exons (see FIG. 4).

For example, an oligonucleotide pair of primers specifically hybridizable with variant as depicted in SEQ ID NO:1, is set forth in SEQ ID NO:15 and 16, or 17 and 18; or 19 and 20. Another example, an oligonucleotide pair of primers specifically hybridizable with variants as depicted in SEQ ID NOs:2 and 3, is set forth in 17 and 18; or 19 and 20.

As is described hereinabove, the UbcH10 variants of the present invention include a unique amino acid sequence (e.g., SEQ ID NO:7) while lacking the conserved E2 catalytic site (i.e., the catalytic Cysteine) which mediates ubiquitin-thiolester formation.

It is well established that dominant negative sequences of UbcH10 which carry a mutation in the active site cysteine, induce cell cycle arrest at metaphase as well as inhibit sister chromatid separation and cyclin B degradation (Townsely, et.al., PNAS, 94: 2362-7, 1997), a phenomenon, which may have a strong cytostatic effect [Rolfe, et.al., J. Mol. Med. 75:5-17, 1997].

Thus, without being bound by theory, the present inventors suggest that the variants of the present invention may serve as dominant negative mutants of wild-type UbcH10 since they are devoid of an E2 active site (i.e., UBC domain) and yet maintain an ability to bind E3. In addition, absence of the destruction box from the variants of the present invention may lead to disregulation of these variants during cell cycle since the destruction box targets UbcH10 for autoubiquitination augmented by APC/C, and degradation at late M phase as cells exit from mitosis [Yamanaka, A., et. al., Mol. Biol. Cell 11: 2821-31, 2000; Lin, Y., et al., JBC 277: 21913-21, 2002], further supporting their proposed dominant negative role.

Therefore, according to another aspect of the present invention there is provided a method of treating a UbcH10-related disease in a subject.

The method is effected by specifically upregulating in the subject expression of a UbcH10 polypeptide at least 55% homologous to SEQ ID NO:7 or 8, as determined using the BlastP software of the National Center of Biotechnology Information (NCBI) using default parameters.

According to preferred embodiments of the present invention upregulating expression of the polypeptide is effected by: (i) administering the polypeptide to the subject; (ii) administering an expressible polynucleotide encoding the polypeptide to the subject; (iii) increasing expression of endogenous UbcH10 polypeptide in the subject; (iv) increasing activity of endogenous UbcH10 polypeptide in the subject; (v) introducing at least one substrate of UbcH10 polypeptide to the subject; and/or (vi) administering UbcH10 polypeptide-expressing cells into the subject.

Preferably, the polynucleotide is as set forth in SEQ ID NO:1, 2 or 3 and the UbcH10 polypeptide is as set forth in SEQ ID NO:4, 5 or 6.

It will be appreciated that the inhibitory properties (e.g., cell-cycle arrest) of the UbcH10 polypeptides of the present invention can be used in a number of therapeutic applications. In such applications it is highly desirable to employ the minimal and most efficacious peptide regions, which still exert inhibitory function. Identification of such peptide regions can be effected using various approaches, including, for example, display techniques as previously described.

Without being bound to any theory, since as is described in Examples 1-6 of the Examples section which follows the UbcH10 splice variants of the present invention are overexpressed in various lung and ovary cancers, such splice variants can represent inducers of tumor formation and/or progression.

Thus, according to yet another aspect of the present invention the present invention also envisages the use of agents capable of downregulating the UbcH10 splice variants of the present invention in treatment of diseases associated with overexpression of the UbcH10 splice variants of the present invention.

Thus, according to yet another aspect of the present invention there is provided a method of treating UbcH10-related disease in a subject.

The method is effected by specifically downregulating in the subject expression level and/or activity of a UbcH10 polypeptide at least 55% homologous to SEQ ID NO:7 or 8, as determined using the BlastP software of the National Center of Biotechnology Information (NCBI) using default parameters.

According to preferred embodiments downregulating is effected by introducing into the subject an agent selected from the group consisting of: (a) a molecule which binds the UbcH10 polypeptide; (b) an enzyme which cleaves the UbcH10 polypeptide; (c) an antisense polynucleotide capable of specifically hybridizing with at least part of an mRNA transcript encoding the UbcH10 polypeptide; (d) a ribozyme which specifically cleaves at least part of an mRNA transcript encoding the UbcH10 polypeptide; (e) a small interfering RNA (siRNA) molecule which specifically cleaves at least part of a transcript encoding the UbcH10 polypeptide; (f) a non-functional analogue of at least a catalytic or binding portion of the UbcH10 polypeptide; (g) a molecule which prevents the UbcH10 polypeptide activation or substrate binding.

Preferably, the UbcH10 polypeptide is as set forth in SEQ ID NO:4, 5 or 6.

An example of an agent which can be used along with the present invention to downregulate UbcH10 is an antibody capable of specifically binding the isolated polypeptides of the present invention which are described hereinabove.

Another example of an agent which can specifically downregulate the UbcH10 transcript variants of the present invention is an antisense oligonucleotide such as 5′-CCCACTGCCATGAGGGTCAT (SEQ ID NO:37) which corresponds to nucleic acid 219-238 of SEQ ID NO:2; 5′-TGAGTTTCTTGTTCCAGCTG (SEQ ID NO:38) which corresponds to nucleic acid 307-326 of SEQ ID NO:3; and 5′-GGTCTTCATATACCTGGCAT (SEQ ID NO:39) which corresponds to nucleic acids 409-429 of SEQ ID NO:3.

Another example of an agent which can specifically downregulate the UbcH10 transcript variants of the present invention is an siRNA molecule such as 5′-GCTGGAACAAGAAACTCAAGA (SEQ ID NO:40) which corresponds to nucleic acid 309-329 of SEQ ID NO:3 and/or 5′-GCCAGGTATATGAAGACCT (SEQ ID NO:41) which corresponds to nucleic acid 411-429 of SEQ ID NO:3.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

The markers of the present invention were tested with regard to their expression in various cancerous and non-cancerous tissue samples. A description of the tissue samples used in the testing panels is provided in Tables 3 and 4 below. Tests were then performed as described in the Examples below.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Experimental Procedures

RNA preparation—RNA was obtained from Clontech (Franklin Lakes, N.J. USA 07417, www.clontech.com), BioChain Inst. Inc. (Hayward, Calif. 94545 USA www.biochain.com), ABS (Wilmington, Del. 19801, USA, http://www.absbioreagents.com) or Ambion (Austin, Tex. 78744 USA, http://www.ambion.com). Alternatively, RNA was generated from tissue samples using TRI-Reagent (Molecular Research Center), according to Manufacturer's instructions. Tissue samples were obtained from patients or from postmortem [e.g., Gynecologic Oncology Group (GOG)]. Total RNA samples were treated with DNaseI (Ambion) and purified using RNeasy columns (Qiagen).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units Rnasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

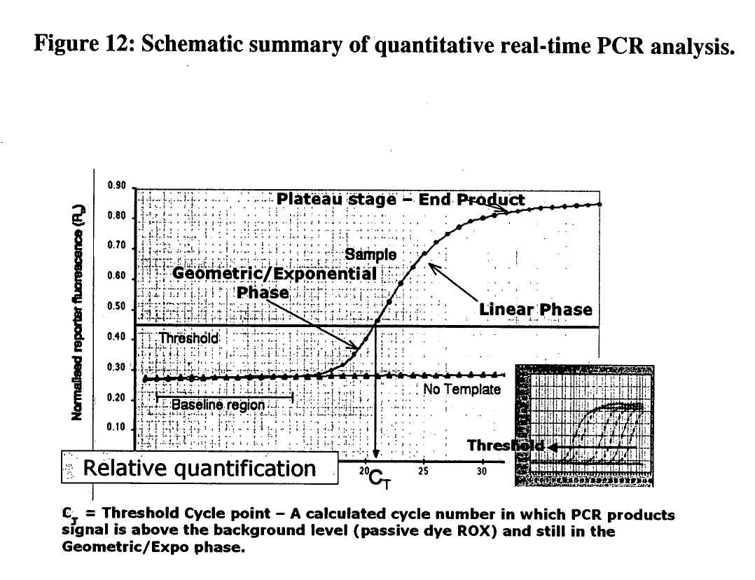

Real-Time RT-PCR analysis—cDNA (5 μl) prepared as described above, was used as a template in Real-Time PCR reactions using the SYBR Green I assay (PE Applied Biosystem) with specific primers and UNG Enzyme (Eurogentech or ABI or Roche). The amplification was effected as follows, 50° C. for 2 min, 95° C. for 10 min, and then 40 cycles of 95° C. for 15 sec, followed by 60° C. for 1 min. Detection was effected using PE Applied Biosystem SDS 7000. The cycle in which the reactions achieved a threshold level (Ct) of fluorescence was registered and served to calculate the relative transcript quantity in the RT reactions. The relative quantity was calculated using the equation Q=efficiencyl-Ct. The efficiency of the PCR reaction was calculated from a standard curve created using serial dilutions of reverse transcription (RT) reactions prepared from RNA purified from 5 cell-lines (HCT116, H1299, DU145, MCF7, ES-2). To minimize inherent differences in the RT reaction, the resulting relative quantities were normalized to the geometric mean of the relative quantities of several housekeeping (HSKP) genes. Note that different HSKP genes were used for the different tissue panels. Schematic summary of quantitative real-time PCR analysis is presented in FIG. 12. As shown, the x-axis shows the cycle number. The CT=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

### Example 1

Expression of UbcH10 transcripts which are detectable by SEQ ID NO:12 in normal and cancerous lung tissues—Expression of the UbcH10 transcripts detectable by SEQ ID NO:12 (an amplicon of a UbcH10 variant as set forth in SEQ ID NO:1; forward primer—SEQ ID NO:15; reverse primer—SEQ ID NO:16) was measured by real time PCR. In parallel the expression of four housekeeping genes—PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), Ubiquitin (GenBank Accession No. BC000449; amplicon—SEQ ID NO:33; forward primer—SEQ ID NO:34; reverse primer—SEQ ID NO:35) and SDHA (GenBank Accession No. NM_{—}004168; amplicon—SEQ ID NO:30; forward primer—SEQ ID NO:31; reverse primer—SEQ ID NO:32), was measured similarly. For each RT sample, the expression of SEQ ID NO:12 was normalized to the geometric mean of the quantities of the housekeeping genes. The normalized quantity of each RT sample was then divided by the averaged quantity of the normal post-mortem (PM) samples (Sample Nos. 47-50, 90-93, 96-99, Table 3, below), to obtain a value of fold up-regulation for each sample relative to averaged normal samples.

FIGS. 5a-b show a histogram and scatter plot, respectively, showing over expression of the above-indicated UbcH10 transcripts in cancerous lung samples relative to the normal samples. The number of samples that exhibit at least 5 fold over-expression, out of the total number of samples tested is indicated below the cancer subtypes in FIG. 5a. The number of samples tested is indicated below each cancer subtype in FIG. 5b. Also provided is the percentage of samples, which exhibited at least 5 fold over-expression.

As is evident from FIGS. 5a-b, the expression of UbcH10 transcripts detectable by SEQ ID NO:12 in cancer samples was significantly higher than in the normal samples (Sample Nos. 46-50, 90-93, 96-99, Table 3). Notably an over-expression of at least 5 fold was found in 8 out of 15 adenocarcinoma, 15 out of 16 squamous, 4 out of 4 large cell, and 8 out of 8 small cell samples.

TABLE 3

| sample rename | Lot No. | source | pathology | Grade | gender/age |
|---|---|---|---|---|---|
| 1-B-Adeno G1 | A504117 | Biochain | Adenocarcinoma | 1 | F/29 |
| 2-B-Adeno G1 | A504118 | Biochain | Adenocarcinoma | 1 | M/64 |
| 95-B-Adeno G1 | A610063 | Biochain | Adenocarcinoma | 1 | F/54 |
| 12-B-Adeno G2 | A504119 | Biochain | Adenocarcinoma | 2 | F/74 |
| 75-B-Adeno G2 | A609217 | Biochain | Adenocarcinoma | 2 | M/65 |
| 77-B-Adeno G2 | A608301 | Biochain | Adenocarcinoma | 2 | M/44 |
| 13-B-Adeno G2-3 | A504116 | Biochain | Adenocarcinoma | 2-3 | M/64 |
| 89-B-Adeno G2-3 | A609077 | Biochain | Adenocarcinoma | 2-3 | M/62 |
| 76-B-Adeno G3 | A609218 | Biochain | Adenocarcinoma | 3 | M/57 |
| 94-B-Adeno G3 | A610118 | Biochain | Adenocarcinoma | 3 | M/68 |
| 3-CG-Adeno | CG-200 | Ichilov | Adenocarcinoma | | NA |
| 14-CG-Adeno | CG-111 | Ichilov | Adenocarcinoma | | M/68 |
| 15-CG-Bronch adeno | CG-244 | Ichilov | Bronchioloalveolar adenocarcinoma | | M/74 |
| 45-B-Alvelous Adeno | A501221 | Biochain | Alveolus carcinoma | | F/50 |
| 44-B-Alvelous Adeno G2 | A501123 | Biochain | Alveolus carcinoma | 2 | F/61 |
| 19-B-Squamous G1 | A408175 | Biochain | Squamous carcinoma | 1 | M/78 |
| 16-B-Squamous G2 | A409091 | Biochain | Squamous carcinoma | 2 | F/68 |
| 17-B-Squamous G2 | A503183 | Biochain | Squamous carcinoma | 2 | M/57 |
| 21-B-Squamous G2 | A503187 | Biochain | Squamous carcinoma | 2 | M/52 |
| 78-B-Squamous G2 | A607125 | Biochain | Squamous Cell Carcinoma | 2 | M/62 |
| 80-B-Squamous G2 | A609163 | Biochain | Squamous Cell Carcinoma | 2 | M/74 |
| 18-B-Squamous G2-3 | A503387 | Biochain | Squamous Cell Carcinoma | 2-3 | M/63 |
| 81-B-Squamous G3 | A609076 | Biochain | Squamous Carcinoma | 3 | m/53 |
| 79-B-Squamous G3 | A609018 | Biochain | Squamous Cell Carcinoma | 3 | M/67 |
| 20-B-Squamous | A501121 | Biochain | Squamous Carcinoma | | M/64 |
| 22-B-Squamous | A503386 | Biochain | Squamous Carcinoma | | M/48 |
| 88-B-Squamous | A609219 | Biochain | Squamous Cell Carcinoma | | M/64 |
| 100-B-Squamous | A409017 | Biochain | Squamous Carcinoma | | M/64 |
| 23-CG-Squamous | CG-109 (1) | Ichilov | Squamous Carcinoma | | M/65 |
| 24-CG-Squamous | CG-123 | Ichilov | Squamous Carcinoma | | M/76 |
| 25-CG-Squamous | CG-204 | Ichilov | Squamous Carcinoma | | M/72 |
| 87-B-Large cell G3 | A609165 | Biochain | Large Cell Carcinoma | 3 | F/47 |
| 38-B-Large cell | A504113 | Biochain | Large cell | | M/58 |
| 39-B-Large cell | A504114 | Biochain | Large cell | | F/35 |
| 82-B-Large cell | A609170 | Biochain | Large Cell Neuroendocrine | | M/68 |
| | | | Carcinoma | | |
| 30-B-Small cell carci G3 | A501389 | Biochain | small cell | 3 | M/34 |
| 31-B-Small cell carci G3 | A501390 | Biochain | small cell | 3 | F/59 |
| 32-B-Small cell carci G3 | A501391 | Biochain | small cell | 3 | M/30 |
| 33-B-Small cell carci G3 | A504115 | Biochain | small cell | 3 | M |
| 86-B-Small cell carci G3 | A608032 | Biochain | Small Cell Carcinoma | 3 | F/52 |
| 83-B-Small cell carci | A609162 | Biochain | Small Cell Carcinoma | | F/47 |
| 84-B-Small cell carci | A609167 | Biochain | Small Cell Carcinoma | | F/59 |
| 85-B-Small cell carci | A609169 | Biochain | Small Cell Carcinoma | | M/66 |
| 46-B-N M44 | A501124 | Biochain | Normal M44 | | F/61 |
| 47-B-N | A503205 | Biochain | Normal PM | | M/26 |
| 48-B-N | A503206 | Biochain | Normal PM | | M/44 |
| 49-B-N | A503384 | Biochain | Normal PM | | M/27 |
| 50-B-N | A503385 | Biochain | Normal PM | | M/28 |
| 90-B-N | A608152 | Biochain | Normal (Pool 2) PM | | pool 2 |
| 91-B-N | A607257 | Biochain | Normal (Pool 2) PM | | pool 2 |
| 92-B-N | A503204 | Biochain | Normal PM | | m/28 |
| 93-Am-N | 111P0103A | Ambion | Normal PM | | F/61 |
| 96-Am-N | 36853 | Ambion | Normal PM | | F/43 |
| 97-Am-N | 36854 | Ambion | Normal PM | | M/46 |
| 98-Am-N | 36855 | Ambion | Normal PM | | F/72 |
| 99-Am-N | 36856 | Ambion | Normal PM | | M/31 |

According to the present invention, UbcH10 transcripts detectable by SEQ ID NO:12 (e.g., variant as set forth in SEQ ID NO:1) are a non-limiting example of a marker for diagnosing lung cancer. The above UbcH10 variant marker of the present invention can be used alone or in combination, for various uses, including but not limited to, prognosis, prediction, screening, early diagnosis, staging, determination of cancer origin in organs which are different from lung, therapy selection and treatment monitoring of lung cancer. Although optionally any method may be used to detect overexpression and/or differential expression of this marker, preferably a NAT-based technology is used. Therefore, optionally and preferably, any nucleic acid molecule capable of selectively hybridizing to UbcH10 transcripts detectable by SEQ ID NO:12 (e.g., variant as set forth in SEQ ID NO:1) as previously defined is also encompassed within the present invention. Primer pairs are also optionally and preferably encompassed within the present invention; for example, for the above experiment, the following primer pair was used as a non-limiting illustrative example only of a suitable primer pair: SEQ ID NO:12 (amplicon of SEQ ID NO:1); forward primer (SEQ ID NO:15): TTTTCAAATGGGTAGGGACCATC; and reverse primer (SEQ ID NO:16): TGAGTTTCTCTGGGACCGGA.

The present invention also preferably encompasses any amplicon obtained through the use of any suitable primer pair; for example, for the above experiment, the following amplicon was obtained as a non-limiting illustrative example only of a suitable amplicon: amplicon (SEQ ID NO:12): TTTTCAAATGGGTAGGGAC CATCCATGGAGCAGCTGGAACAGCAGTGGGGAGCATCAGAACCAGCTCA ACAGTTTGTCTACTGTCCGGTCCCAGAGAAACTCA

According to other preferred embodiments of the present invention, UbcH10 transcripts detectable by SEQ ID NO:12 (e.g., variant as set forth in SEQ ID NO:1) or a fragment thereof comprises a biomarker for detecting lung cancer. Optionally and more preferably, the fragment of UbcH10 transcript detectable by SEQ ID NO:12 (e.g., variant as set forth in SEQ ID NO:1) comprises segment_{—}19 (SEQ ID NO:9; which is contained in SEQ ID NOs:1 and 2). Optionally and more preferably, the fragment of UbcH10 transcript detectable by SEQ ID NO:12 (e.g., variant as set forth in SEQ ID NO:1) comprises segment_{—}20 (SEQ ID NO:10, which is contained in SEQ ID NOs:1, 2 and 3). Also optionally and more preferably, any suitable method may be used for detecting a fragment such as segment_{—}19 for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to SEQ ID NO:1 as described above, including but not limited to SEQ ID NOs:4 and 7. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

The present invention also optionally and preferably encompasses any nucleic acid sequence or fragment thereof, or amino acid sequence or fragment thereof, corresponding to UbcH10 transcripts detectable by SEQ ID NO:12, as described above, optionally for any application.

### Example 2

Expression of UbcH10 transcripts which are detectable by SEQ ID NO:12 in normal, benign and cancerous ovary tissues—Expression of transcripts detected by SEQ ID NO:12 (such as transcripts as set forth in SEQ ID NO:1) was measured by real time PCR. In parallel the expression of four housekeeping genes—PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), GAPDH (GenBank Accession No. BC026907; amplicon—SEQ ID NO:27; forward primer—SEQ ID NO:28; reverse primer—SEQ ID NO:29) and SDHA (GenBank Accession No. NM_{—}004168; amplicon—SEQ ID NO:30; forward primer—SEQ ID NO:31; reverse primer—SEQ ID NO:32), was measured similarly. For each RT sample, the expression of SEQ ID NO:12 was normalized to the geometric mean of the quantities of the housekeeping genes. The normalized quantity of each RT sample was then divided by the averaged quantity of the normal post-mortem samples (see samples numbers 45-48, 71, Table 4 below) to obtain a value of fold up-regulation of each sample relative to averaged normal samples.

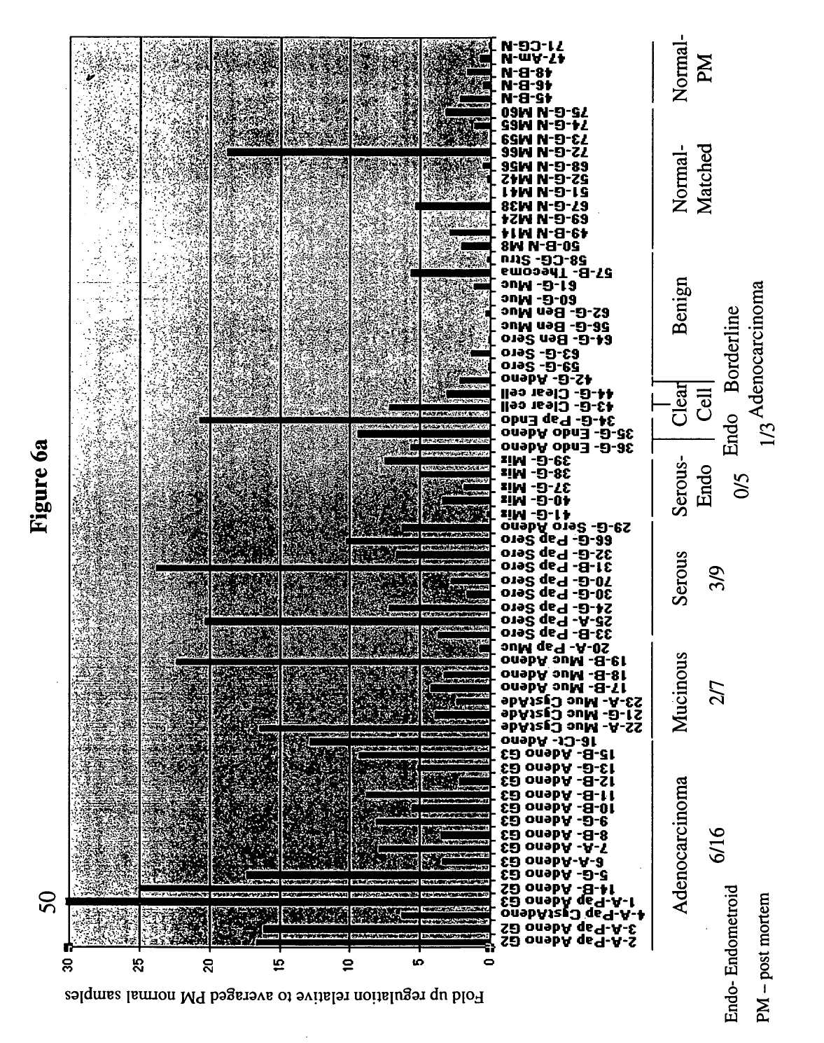

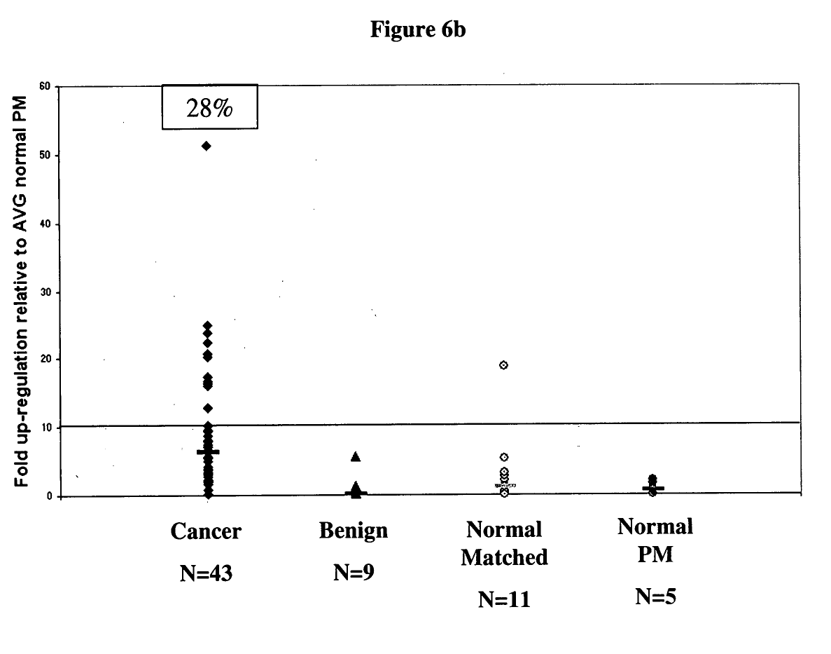

FIGS. 6a-b are a histogram and scatter plot, respectively, showing over expression of the above-indicated UbcH10 transcripts in cancerous and benign ovarian samples relative to the normal post-mortem (PM) samples. The number of samples that exhibit at least 10 fold over-expression, out of the total number of samples tested is indicated below the cancer subtypes in FIG. 6a. The number of samples tested is indicated below each cancer subtype in FIG. 6b. Also provided is the percentage of samples which showed at least 10 fold over-expression.

As is evident from FIGS. 6a-b, the expression of UbcH10 transcripts detectable by SEQ ID NO:12 in cancer samples was significantly higher than in the normal samples (samples no. 45-52, 67-69, 71-75, Table 4) and benign samples (samples 56-64, Table 4). Notably, over-expression of transcripts detectable by SEQ ID NO:12 of at least 10 fold was found in 6 out of 16 adenocarcinoma, 2 out of 7 Mucinus adenocarcinoma, 3 out of 9 Serous adenocarcinoma, and in 1 out of 3 endometroid adenocarcinoma samples.

TABLE 4

| Sample name | Lot number | Source | Tissue | Pathology | Grade | gender/age |
|---|---|---|---|---|---|---|
| 2-A-Pap Adeno G2 | ILS-1408 | ABS | ovary | Papillary adenocarcinoma | 2 | 53/F |
| 3-A-Pap Adeno G2 | ILS-1431 | ABS | ovary | Papillary adenocarcinoma | 2 | 52/F |
| 4-A-Pap | ILS-7286 | ABS | ovary | Papillary cystadenocarcinoma | 2 | 50/F |
| CystAdeno G2 | | | | | | |
| 1-A-Pap Adeno G3 | ILS-1406 | ABS | ovary | Papillary adenocarcinoma | 3 | 73/F |
| 14-B-Adeno G2 | A501111 | BioChain | ovary | Adenocarcinoma | 2 | 41/F |
| 5-G-Adeno G3 | 99-12-G432 | GOG | ovary | Adenocarcinoma (Stage3C) | 3 | 46/F |
| 6-A-Adeno G3 | A0106 | ABS | ovary | adenocarcinoma | 3 | 51/F |
| 7-A-Adeno G3 | IND-00375 | ABS | ovary | adenocarcinoma | 3 | 59/F |
| 8-B-Adeno G3 | A501113 | BioChain | ovary | adenocarcinoma | 3 | 60/F |
| 9-G-Adeno G3 | 99-06-G901 | GOG | ovary | Adenocarcinoma (maybe serous) | 3 | 84/F |
| 10-B-Adeno G3 | A407069 | Biochain | ovary | Adenocarcinoma | 3 | 60/F |
| 11-B-Adeno G3 | A407068 | Biochain | ovary | Adenocarcinoma | 3 | 49/F |
| 12-B-Adeno G3 | A406023 | Biochain | ovary | Adenocarcinoma | 3 | 45/F |
| 13-G-Adeno G3 | 94-05-7603 | GOG | right ovary | Metastasis adenocarcinoma | 3 | 67/F |
| 15-B-Adeno G3 | A407065 | BioChain | ovary | Carcinoma | 3 | 27/F |
| 16-Ct-Adeno | 1090387 | Clontech | ovary | Carcinoma NOS | | F |
| 22-A-Muc | A0139 | ABS | ovary | Mucinous cystadenocarcinoma | 2 | 72/F |
| CystAde G2 | | | | (Stage1C) | | |
| 21-G-Muc | 95-10-G020 | GOG | ovary | Mucinous cystadenocarcinoma | 2-3 | 44/F |
| CystAde G2-3 | | | | (Stage2) | | |
| 23-A-Muc | VNM-00187 | ABS | ovary | Mucinous cystadenocarcinoma with | 3 | 45/F |
| CystAde G3 | | | | low malignant | | |
| 17-B-Muc Adeno | A504084 | BioChain | ovary | Mucinous adenocarcinoma | 3 | 51/F |
| G3 | | | | | | |
| 18-B-Muc Adeno | A504083 | BioChain | ovary | Mucinous adenocarcinoma | 3 | 45/F |
| G3 | | | | | | |
| 19-B-Muc Adeno | A504085 | BioChain | ovary | Mucinous adenocarcinoma | | 34/F |
| G3 | | | | | | |
| 20-A-Pap Muc | USA-00273 | ABS | ovary | Papillary mucinous | | 45/F |
| CystAde | | | | cystadenocarcinoma | | |
| 33-B-Pap Sero | A503175 | BioChain | ovary | Serous papillary | 1 | 41/F |
| CystAde G1 | | | | cystadenocarcinoma | | |
| 25-A-Pap Sero | N0021 | ABS | ovary | Papillary serous adenocarcinoma | 3 | 55/F |
| Adeno G3 | | | | (StageT3CN1MX) | | |
| 24-G-Pap Sero | 2001-07-G801 | GOG | ovary | Papillary serous adenocarcinoma | 3 | 68/F |
| Adeno G3 | | | | | | |
| 30-G-Pap Sero | 2001-08-G011 | GOG | ovary | Papillary serous carcinoma | 3 | 72/F |
| Adeno G3 | | | | (Stage1C) | | |
| 70-G-Pap Sero | 95-08-G069 | GOG | ovary | Papillary serous adenocarcinoma | 3 | F |
| Adeno G3 | | | | | | |
| 31-B-Pap Sero | A503176 | BioChain | ovary | Serous papillary | 3 | 52/F |
| CystAde G3 | | | | cystadenocarcinoma | | |
| 32-G-Pap Sero | 93-09-4901 | GOG | ovary | Serous papillary | 3 | F |
| CystAde G3 | | | | cystadenocarcinoma | | |
| 66-G-Pap Sero | 2000-01-G413 | GOG | ovary | Papillary serous carcinoma | | F |
| Adeno G3 SIV | | | | (metastais of primary peritoneum) | | |
| | | | | (Stage4) | | |
| 29-G-Sero Adeno G3 | 2001-12-G035 | GOG | right ovary | Serous adenocarcinoma (Stage3A) | 3 | 50/F |
| 41-G-Mix | 98-03-G803 | GOG | ovary | Mixed epithelial | 2 | 38 |
| Sero/Muc/Endo | | | | cystadenocarcinoma with | | |
| G2 | | | | mucinous, endometrioid, squamous | | |
| | | | | and papillary serous (Stage2) | | |
| 40-G-Mix | 95-11-G006 | GOG | ovary, endometrium | Papillary serous and endometrioid | 2 | 49/F |
| Sero/Endo G2 | | | | cystadenocarcinoma (Stage3C) | | |
| 37-G-Mix | 2002-05-G513 | GOG | ovary | Mixed serous and endometrioid | 3 | 56/F |
| Sero/Endo G3 | | | | adenocarcinoma | | |
| 38-G-Mix | 2002-05-G509 | GOG | ovary | Mixed serous and endometrioid | 3 | 64/F |
| Sero/Endo G3 | | | | adenocarcinoma of mullerian | | |
| | | | | (Stage3C) | | |
| 39-G-Mix | 2001-12-G037 | GOG | ovary | Mixed serous and endometrioid | 3 | F |
| Sero/Endo G3 | | | | adenocarcinoma | | |
| 36-G-Endo Adeno | 2000-09-G621 | GOG | ovary | Endometrial adenocarcinoma | 1-2 | 69/F |
| G1-2 | | | | | | |
| 35-G-Endo Adeno | 94-08-7604 | GOG | right ovary | Endometrioid adenocarcinoma | 2 | 39/F |
| G2 | | | | | | |
| 34-G-Pap Endo | 95-04-2002 | GOG | ovary | Papillary endometrioid | 3 | 68/F |
| Adeno G3 | | | | adenocarcinoma (Stage3C) | | |
| 43-G-Clear cell | 2001-10-G002 | GOG | ovary | Clear cell adenocarcinoma | 3 | 74/F |
| Adeno G3 | | | | | | |
| 44-G-Clear cell | 2001-07-G084 | GOG | ovary | Clear cell adenocarcinoma | | 73/F |
| Adeno | | | | (Stage3A) | | |
| 42-G-Adeno | 98-08-G001 | GOG | ovary | Epithelial adenocarcinoma of | | 46/F |
| borderline | | | | borderline malignancy | | |
| 59-G-Sero | 98-12-G401 | GOG | ovary | Serous CysAdenoFibroma | | 77/F |
| CysAdenoFibroma | | | | | | |
| 63-G-Sero | 2000-10-G620 | GOG | ovary | Serous CysAdenoFibroma of | | 71/F |
| CysAdenoFibroma | | | | borderline malignancy | | |
| 64-G-Ben Sero | 99-06-G039 | GOG | ovary | Bengin Serous CysAdenoma | | 57/F |
| CysAdenoma | | | | | | |
| 56-G-Ben Muc | 99-01-G407 | GOG | left ovary | Bengin mucinus cysadenoma | | 46/F |
| CysAdeno | | | | | | |
| 62-G-Ben Muc | 99-10-G442 | GOG | ovary | Bengin mucinus cysadenoma | | 32/F |
| CysAdenoma | | | | | | |
| 60-G-Muc | 99-01-G043 | GOG | ovary | Mucinous Cysadenoma | | 40/F |
| CysAdenoma | | | | | | |
| 61-G-Muc | 99-07-G011 | GOG | ovary | Mucinous Cysadenoma | | 63/F |
| CysAdenoma | | | | | | |
| 57-B-Thecoma | A407066 | BioChain | ovary | Thecoma | | 56/F |
| 58-CG-Stru | CG-177 | Ichilov | ovary | Struma ovary/monodermal | | 58/F |
| teratoma | | | | teratoma | | |
| 50-B-N M8 | A501114 | BioChain | ovary | Normal (matched tumor A501113) | | 60/F |
| 49-B-N M14 | A501112 | BioChain | ovary | Normal (matched tumor A501111) | | 41/F |
| 69-G-N M24 | 2001-07- | GOG | ovary | Normal (matched tumor 2001-07- | | 68/F |
| | G801N | | | G801) | | |
| 67-G-N M38 | 2002-05-509N | GOG | ovary | Normal (matched tumor 2002-05- | | 64/F |
| | | | | G509) | | |
| 51-G-N M41 | 98-03-G803N | GOG | ovary | Normal (matched tumor 98-03- | | 38/F |
| | | | | G803) | | |
| 52-G-N M42 | 98-08-G001N | GOG | ovary | Normal (matched tumor 98-08- | | 46/F |
| | | | | G001) | | |
| 68-G-N M56 | 99-01-G407N | GOG | ovary | Normal (matched bengin 99-01- | | 46/F |
| | | | | G407) | | |
| 72-G-N M66 | 2000-01- | GOG | ovary | Normal (matched tumor 2000-01- | | F |
| | G413N | | | G413) | | |
| 73-G-N M59 | 98-12-G401N | GOG | ovary | Normal (matched tumor 98-12- | | 77/F |
| | | | | G401) | | |
| 74-G-N M65 | 97-11-G320N | GOG | ovary | Normal (matched tumor 97- | | 41/F |
| | | | | 11G320) | | |
| 75-G-N M60 | 99-01-G043N | GOG | ovary | Normal (matched tumor 99-01- | | 40/F |
| | | | | G043) | | |
| 45-B-N | A503274 | BioChain | ovary | Normal PM | | 41/F |
| 46-B-N | A504086 | BioChain | ovary | Normal PM | | 41/F |
| 48-B-N | A504087 | BioChain | ovary | Normal PM | | 51/F |
| 47-Am-N | 061P43A | Ambion | ovary | Normal PM | | 16/F |
| 71-CG-N | CG-188-7 | Ichilov | ovary | Normal PM | | 49/F |

According to the present invention, UbcH10 transcripts detectable by SEQ ID NO:12 (e.g., variant as set forth in SEQ ID NO:1) are a non-limiting example of a marker for diagnosing ovarian cancer. The above UbcH10 variant marker of the present invention can be used alone or in combination, for various uses, including but not limited to, prognosis, prediction, screening, early diagnosis, staging, determination of cancer origin in organs which are different from ovary, therapy selection and treatment monitoring of ovarian cancer. Although optionally any method may be used to detect overexpression and/or differential expression of this marker, preferably a NAT-based technology is used. Therefore, optionally and preferably, any nucleic acid molecule capable of selectively hybridizing to UbcH10 transcripts detectable by SEQ ID NO:12 (e.g., variant as set forth in SEQ ID NO:1) as previously defined is also encompassed within the present invention. Primer pairs are also optionally and preferably encompassed within the present invention; for example, for the above experiment, the following primer pair was used as a non-limiting illustrative example only of a suitable primer pair; SEQ ID NO:12 (amplicon of SEQ ID NO:1) was amplified using the -forward primer (SEQ ID NO:15): TTTTCAAATGGGTAGGGACCATC; and reverse primer (SEQ ID NO:16): TGAGTTTCTCTGGGACCGGA.

The present invention also preferably encompasses any amplicon obtained through the use of any suitable primer pair; for example, for the above experiment, the following amplicon was obtained as a non-limiting illustrative example only of a suitable amplicon: amplicon (SEQ ID NO:12): TTTTCAAATGGGTAG GGACCATCCATGGAGCAGCTGGAACAGCAGTGGGGAGCATCAGAACCAG CTCAACAGTTTGTCTACTGTCCGGTCCCAGAGAAACTCA

According to other preferred embodiments of the present invention, UbcH10 transcripts detectable by SEQ ID NO:12 (e.g., variant as set forth in SEQ ID NO:1) or a fragment thereof comprises a biomarker for detecting ovarian cancer. Optionally and more preferably, the fragment of UbcH10 transcript detectable by SEQ ID NO:12 (e.g., variant as set forth in SEQ ID NO:1) comprises segment_{—}19 (SEQ ID NO:9; which is contained in SEQ ID NOs:1 and 2). Optionally and more preferably, the fragment of UbcH10 transcript detectable by SEQ ID NO:12 (e.g., variant as set forth in SEQ ID NO:1) comprises segment_{—}20 (SEQ ID NO:10, which is contained in SEQ ID NOs:1, 2 and 3). Also optionally and more preferably, any suitable method may be used for detecting a fragment such as segment_{—}19 for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to SEQ ID NO:1 as described above, including but not limited to SEQ ID NOs:4 and 7. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

The present invention also optionally and preferably encompasses any nucleic acid sequence or fragment thereof, or amino acid sequence or fragment thereof, corresponding to UbcH10 transcripts detectable by SEQ ID NO:12, as described above, optionally for any application.

### Example 3

Expression of UbcH10—transcripts which are detectable by SEQ ID NO:13 in normal and cancerous lung tissues—Expression of transcripts detected by SEQ ID NO:13 (i.e., an amplicon of transcripts as set forth in SEQ ID NOs:1 and 2; forward primer—SEQ ID NO:17; reverse primer—SEQ ID NO:18) was measured by real time PCR. In addition the expression of four housekeeping genes—PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), Ubiquitin (GenBank Accession No. BC000449; amplicon—SEQ ID NO:33; forward primer—SEQ ID NO:34; reverse primer—SEQ ID NO:35) and SDHA (GenBank Accession No. NM_{—}004168; amplicon—SEQ ID NO:30; forward primer—SEQ ID NO:31; reverse primer—SEQ ID NO:32), was measured by real time PCR. In each RT sample, the expression of SEQ ID NO:13 was normalized to the geometric mean of the quantities of the housekeeping genes. The normalized quantity of each RT sample was then divided by the averaged quantity of the normal post-mortem (PM) samples (no. 47-50, 90-93, 96-99, Table 3) to obtain a value of fold up-regulation of each sample relative to averaged normal samples.

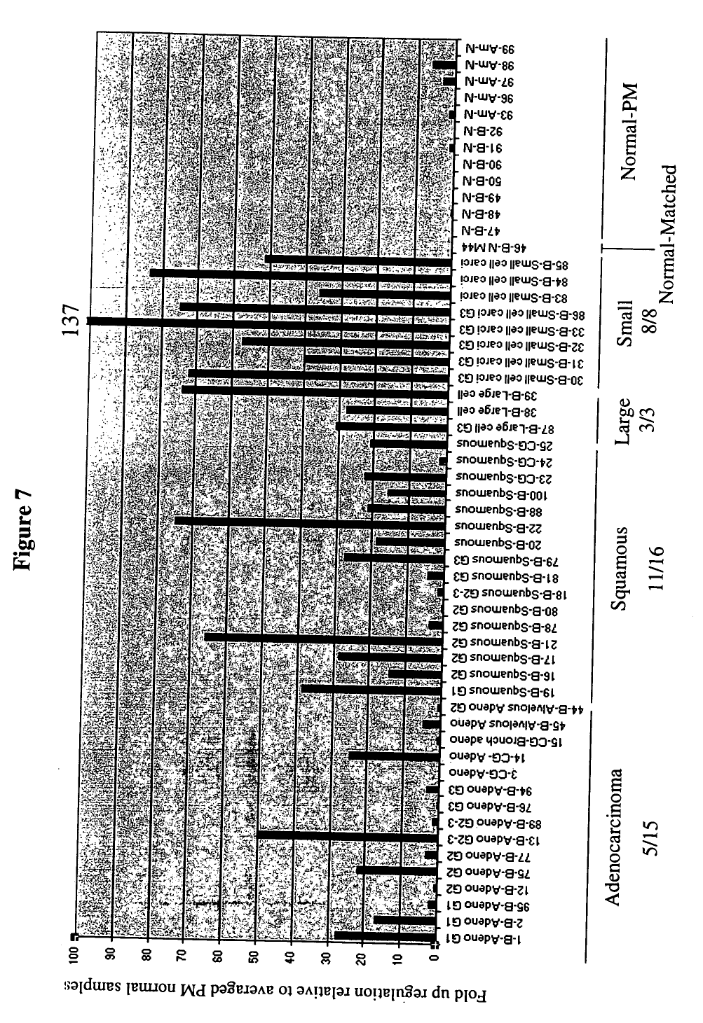

In FIG. 7 the data is shown in a histogram. The number of samples that exhibit at least 10 fold over-expression, out of the total number of samples tested is indicated below the cancer subtypes. As shown in FIG. 7, SEQ ID NO:13 expression in cancer samples was significantly higher than in the normal samples. Notably, SEQ ID NO:13 over-expression of at least 10 fold was found in 5 out of 15 adenocarcinoma, 11 out of 16 squamous, 3 out of 3 large cell, and 8 out of 8 small cell samples.

According to the present invention, UbcH10 transcripts detectable by SEQ ID NO:13 (e.g., variant as set forth in SEQ ID NOs:1 and 2) are a non-limiting example of a marker for diagnosing lung cancer. The above UbcH10 variant marker of the present invention can be used alone or in combination, for various uses, including but not limited to, prognosis, prediction, screening, early diagnosis, staging, determination of cancer origin in organs which are different from lung, therapy selection and treatment monitoring of lung cancer. Although optionally any method may be used to detect overexpression and/or differential expression of this marker, preferably a NAT-based technology is used. Therefore, optionally and preferably, any nucleic acid molecule capable of selectively hybridizing to UbcH10 transcripts detectable by SEQ ID NO:13 (e.g., variant as set forth in SEQ ID NOs:1 and 2) as previously defined is also encompassed within the present invention. Primer pairs are also optionally and preferably encompassed within the present invention; for example, for the above experiment, the following primer pair was used as a non-limiting illustrative example only of a suitable primer pair: SEQ ID NO:13 (amplicon of SEQ ID NOs:1 and 2); forward primer (SEQ ID NO:17): TGTTTCTCCAAATGCCAGAACC; and reverse primer (SEQ ID NO:18): GGCTGGTGACCTGCTTTGA.

The present invention also preferably encompasses any amplicon obtained through the use of any suitable primer pair; for example, for the above experiment, the following amplicon was obtained as a non-limiting illustrative example only of a suitable amplicon: amplicon (SEQ ID NO:13): TGTTTCTCCAAATGCCAG AACCCAACATTGATAGTCCCTTGAACACACATGCTGCCGAGCTCTGGAAA AACCCCACAGCTTTTAAGAAGTACCTGCAAGAAACCTACTCAAAGCAGGT CACCAGCC

According to other preferred embodiments of the present invention, UbcH10 transcripts detectable by SEQ ID NO:13 (e.g., variant as set forth in SEQ ID NOs:1 and 2) or a fragment thereof comprises a biomarker for detecting lung cancer. Optionally and more preferably, the fragment of UbcH10 transcript detectable by SEQ ID NO:13 (e.g., variant as set forth in SEQ ID NOs:1 and 2) comprises segment_{—}19 (SEQ ID NO:9; which is contained in SEQ ID NOs:1 and 2). Optionally and more preferably, the fragment of UbcH10 transcript detectable by SEQ ID NO:13 (e.g., variant as set forth in SEQ ID NOs:1 and 2) comprises segment_{—}20 (SEQ ID NO:10, which is contained in SEQ ID NOs:1, 2 and 3). Also optionally and more preferably, any suitable method may be used for detecting a fragment such as segment_{—}19 for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to SEQ ID NO:1 as described above, including but not limited to SEQ ID NOs:4 and 7. According to still other preferred embodiments, the present invention optionally and preferably encompasses any amino acid sequence or fragment thereof encoded by a nucleic acid sequence corresponding to SEQ ID NO:2 as described above, including but not limited to SEQ ID NOs:5 and 7. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

The present invention also optionally and preferably encompasses any nucleic acid sequence or fragment thereof, or amino acid sequence or fragment thereof, corresponding to UbcH10 transcripts detectable by SEQ ID NO:13, as described above, optionally for any application.

### Example 4

Expression of UbcH10 transcripts which are detected by SEQ ID NO:13 in normal, benign and cancerous ovary tissues—Expression of transcripts detected by SEQ ID NO:13, such as transcripts as set forth in SEQ ID NOs:1 and 2, was measured by real time PCR. In addition, the expression of four housekeeping genes—PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), GAPDH (GenBank Accession No. BC026907; amplicon—SEQ ID NO:27; forward primer—SEQ ID NO:28; reverse primer—SEQ ID NO:29) and SDHA (GenBank Accession No. NM_{—}004168; amplicon—SEQ ID NO:30; forward primer—SEQ ID NO:31; reverse primer—SEQ ID NO:32), was measured similarly. For each RT sample, the expression of SEQ ID NO:13 was normalized to the geometric mean of the quantities of the housekeeping genes. The normalized quantity of each RT sample was then divided by the averaged quantity of the normal post-mortem samples (Samples numbers 45-48, 71, Table 4 supra) to obtain a value of fold up-regulation of each sample relative to averaged normal samples.

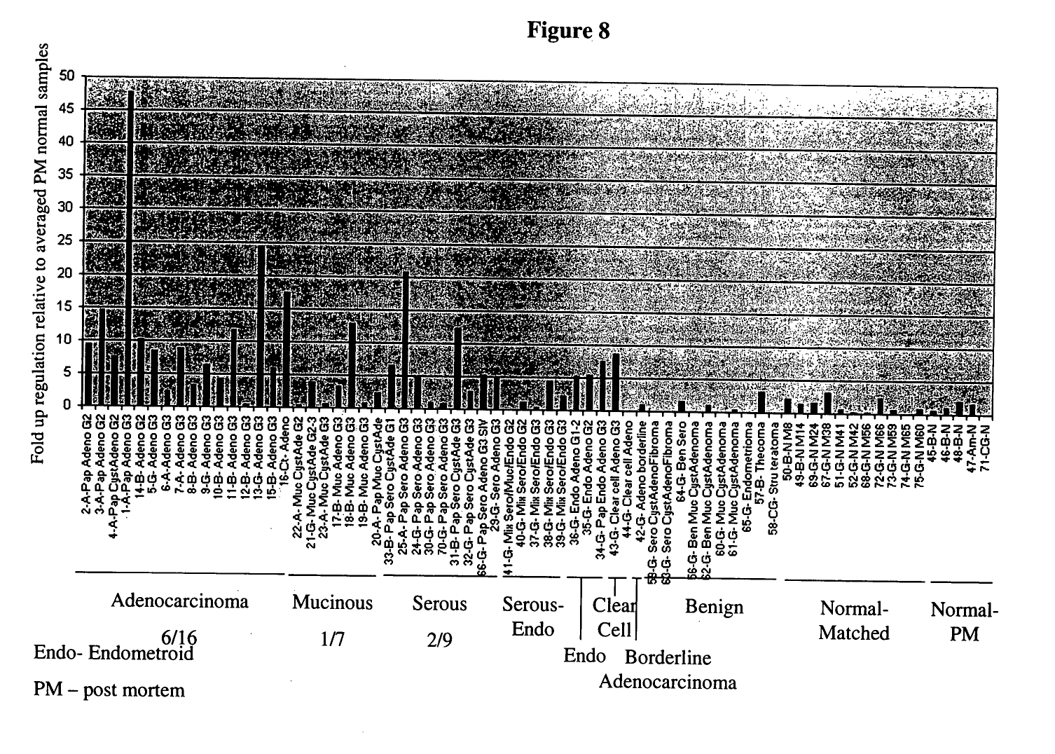

In FIG. 8 data is shown in a histogram presentation. The number of samples that exhibit at least 10 fold over-expression, out of the total number of samples tested is indicated below the cancer subtypes.

As shown in FIG. 8, over-expression of at least 10 fold was detected for SEQ ID NO:13 in 6 out of 16 adenocacinoma, 1 out of 7 Mucinus adenocarcinoma, and in 2 out of 9 Serous adenocarcinoma.

According to the present invention, UbcH10 transcripts detectable by SEQ ID NO:13 (e.g., variant as set forth in SEQ ID NOs:1 and 2) are a non-limiting example of a marker for diagnosing ovarian cancer. The above UbcH10 variant marker of the present invention can be used alone or in combination, for various uses, including but not limited to, prognosis, prediction, screening, early diagnosis, staging, determination of cancer origin in organs which are different from ovary, therapy selection and treatment monitoring of ovarian cancer. Although optionally any method may be used to detect overexpression and/or differential expression of this marker, preferably a NAT-based technology is used. Therefore, optionally and preferably, any nucleic acid molecule capable of selectively hybridizing to UbcH10 transcripts detectable by SEQ ID NO:13 (e.g., variant as set forth in SEQ ID NOs:1 and 2) as previously defined is also encompassed within the present invention. Primer pairs are also optionally and preferably encompassed within the present invention; for example, for the above experiment, the following primer pair was used as a non-limiting illustrative example only of a suitable primer pair: SEQ ID NO:13 (amplicon of SEQ ID NOs:1 and 2); forward primer (SEQ ID NO:17): TGTTTCTCCAAATGCCAGAACC; and reverse primer (SEQ ID NO:18): GGCTGGTGACCTGCTTTGA.

The present invention also preferably encompasses any amplicon obtained through the use of any suitable primer pair; for example, for the above experiment, the following amplicon was obtained as a non-limiting illustrative example only of a suitable amplicon: amplicon (SEQ ID NO:13): TGTTTCTCCAAATG CCAGAACCCAACATTGATAGTCCCTTGAACACACATGCTGCCGAGCTCTG GAAAAACCCCACAGCTTTTAAGAAGTACCTGCAAGAAACCTACTCAAAGC AGGTCACCAGCC

According to other preferred embodiments of the present invention, UbcH10 transcripts detectable by SEQ ID NO:13 (e.g., variant as set forth in SEQ ID NOs:1 and 2) or a fragment thereof comprises a biomarker for detecting ovarian cancer. Optionally and more preferably, the fragment of UbcH10 transcript detectable by SEQ ID NO:13 (e.g., variant as set forth in SEQ ID NOs:1 and 2) comprises segment_{—}19 (SEQ ID NO:9; which is contained in SEQ ID NOs:1 and 2). Optionally and more preferably, the fragment of UbcH10 transcript detectable by SEQ ID NO:13 (e.g., variant as set forth in SEQ ID NOs:1 and 2) comprises segment_{—}20 (SEQ ID NO:10, which is contained in SEQ ID NOs:1, 2 and 3). Also optionally and more preferably, any suitable method may be used for detecting a fragment such as segment_{—}19 for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to SEQ ID NO:1 as described above, including but not limited to SEQ ID NOs:4 and 7. According to still other preferred embodiments, the present invention optionally and preferably encompasses any amino acid sequence or fragment thereof encoded by a nucleic acid sequence corresponding to SEQ ID NO:2 as described above, including but not limited to SEQ ID NOs:5 and 7. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

The present invention also optionally and preferably encompasses any nucleic acid sequence or fragment thereof, or amino acid sequence or fragment thereof, corresponding to UbcH10 transcripts detectable by SEQ ID NO:13, as described above, optionally for any application.

### Example 5

Expression of UbcH10 transcripts which are detectable by SEQ ID NO:14 in normal and cancerous lung tissues—Expression of transcripts detected by SEQ ID NO:14 (i.e., an amplicon of UbcH10 transcripts as set forth in SEQ ID NOs:1 and 2; forward primer—SEQ ID NO:19; reverse primer—SEQ ID NO:20) was measured by real time PCR. In addition the expression of four housekeeping genes—PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), Ubiquitin (GenBank Accession No. BC000449; amplicon—SEQ ID NO:33; forward primer—SEQ ID NO:34; reverse primer—SEQ ID NO:35) and SDHA (GenBank Accession No. NM_{—}004168; amplicon—SEQ ID NO:30; forward primer—SEQ ID NO:31; reverse primer—SEQ ID NO:32), was measured in parallel by real time PCR. For each RT sample, the expression of SEQ ID NO:14 was normalized to the geometric mean of the quantities of the housekeeping genes. The normalized quantity of each RT sample was then divided by the averaged quantity of the normal post-mortem (PM) samples (Samples numbers 47-50, 90-93, 96-99, Table 3 supra) to obtain a value of fold up-regulation of each sample relative to averaged normal samples.

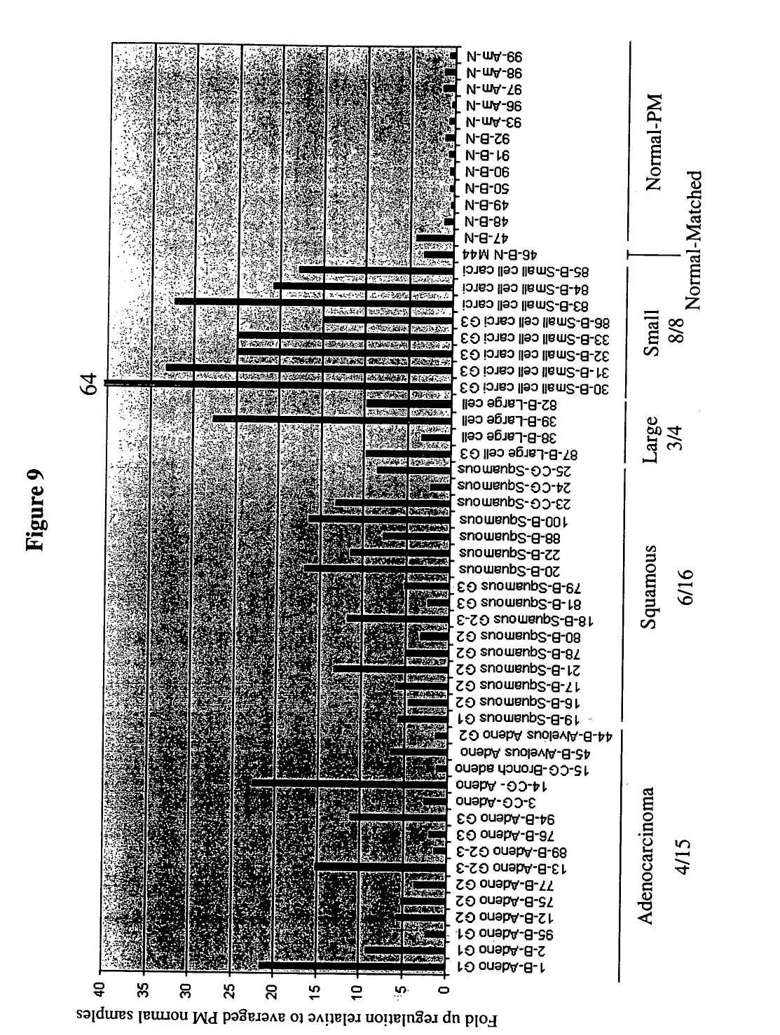

In FIG. 9 data is shown in a histogram presentation. The number of samples that exhibit at least 10 fold over-expression, out of the total number of samples tested is indicated below the cancer subtypes.

As shown in FIG. 9, over-expression of at least 10 fold was found for SEQ ID NO:14 in 4 out of 15 adenocarcinoma, 6 out of 16 squamous, 3 out of 4 large cell, and 8 out of 8 small cell samples.

According to the present invention, UbcH10 transcripts detectable by SEQ ID NO:14 (e.g., variant as set forth in SEQ ID NOs:1 and 2) are a non-limiting example of a marker for diagnosing lung cancer. The above UbcH10 variant marker of the present invention can be used alone or in combination, for various uses, including but not limited to, prognosis, prediction, screening, early diagnosis, staging, determination of cancer origin in organs which are different from lung, therapy selection and treatment monitoring of lung cancer. Although optionally any method may be used to detect overexpression and/or differential expression of this marker, preferably a NAT-based technology is used. Therefore, optionally and preferably, any nucleic acid molecule capable of selectively hybridizing to UbcH10 transcripts detectable by SEQ ID NO:14 (e.g., variant as set forth in SEQ ID NOs:1 and 2) as previously defined is also encompassed within the present invention. Primer pairs are also optionally and preferably encompassed within the present invention; for example, for the above experiment, the following primer pair was used as a non-limiting illustrative example only of a suitable primer pair: SEQ ID NO:14 (amplicon of SEQ ID NOs:1 and 2); forward primer (SEQ ID NO:19): TCTACTGTCCGGTCCCAGAGA; and reverse primer (SEQ ID NO:20): AGTAAGCTCCAGCAGCAGCC.

The present invention also preferably encompasses any amplicon obtained through the use of any suitable primer pair; for example, for the above experiment, the following amplicon was obtained as a non-limiting illustrative example only of a suitable amplicon: amplicon (SEQ ID NO:14): TCTACTGTCCGGTCCCA GAGAAACTCAAGATTCTAGCAAGCCCCTTGTGTGGGGCTTGGGTTGGGAC ATGAGGCTGCTGCTGGAGCTTAC

According to other preferred embodiments of the present invention, UbcH10 transcripts detectable by SEQ ID NO:14 (e.g., variant as set forth in SEQ ID NOs:1 and 2) or a fragment thereof comprises a biomarker for detecting lung cancer. Optionally and more preferably, the fragment of UbcH10 transcript detectable by SEQ ID NO:14 (e.g., variant as set forth in SEQ ID NOs:1 and 2) comprises segment_{—}19 (SEQ ID NO:9; which is contained in SEQ ID NOs:1 and 2). Optionally and more preferably, the fragment of UbcH10 transcript detectable by SEQ ID NO:14 (e.g., variant as set forth in SEQ ID NOs:1 and 2) comprises segment_{—}20 (SEQ ID NO:10, which is contained in SEQ ID NOs:1, 2 and 3). Also optionally and more preferably, any suitable method may be used for detecting a fragment such as segment_{—}19 for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to SEQ ID NO:1 as described above, including but not limited to SEQ ID NOs:4 and 7. According to still other preferred embodiments, the present invention optionally and preferably encompasses any amino acid sequence or fragment thereof encoded by a nucleic acid sequence corresponding to SEQ ID NO:2 as described above, including but not limited to SEQ ID NOs:5 and 7. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide. The present invention also optionally and preferably encompasses any nucleic acid sequence or fragment thereof, or amino acid sequence or fragment thereof, corresponding to UbcH10 transcripts detectable by SEQ ID NO:14, as described above, optionally for any application.

### Example 6

Expression of UbcH10 transcripts which are detectable by SEQ ID NO:14 in normal, benign and cancerous ovary tissues—Expression of transcripts which can be detected by SEQ ID NO:14 (such as transcripts as set forth in SEQ ID NO:1 and 2) was measured by real time PCR. In addition the expression of four housekeeping genes—PBGD (GenBank Accession No. BC019323; amplicon—SEQ ID NO:21; forward primer—SEQ ID NO:22; reverse primer—SEQ ID NO:23), HPRT1 (GenBank Accession No. NM_{—}000194; amplicon—SEQ ID NO:24; forward primer—SEQ ID NO:25; reverse primer—SEQ ID NO:26), GAPDH (GenBank Accession No. BC026907; amplicon—SEQ ID NO:27; forward primer—SEQ ID NO:28; reverse primer—SEQ ID NO:29) and SDHA (GenBank Accession No. NM_{—}004168; amplicon—SEQ ID NO:30; forward primer—SEQ ID NO:31; reverse primer—SEQ ID NO:32), was measured by real time PCR. For each RT sample, the expression of SEQ ID NO:14 was normalized to the geometric mean of the quantities of the housekeeping genes. The normalized quantity of each RT sample was then divided by the averaged quantity of the normal post-mortem samples (Samples numbers 45-48, 71, Table 4) to obtain a value of fold up-regulation of each sample relative to averaged normal samples.

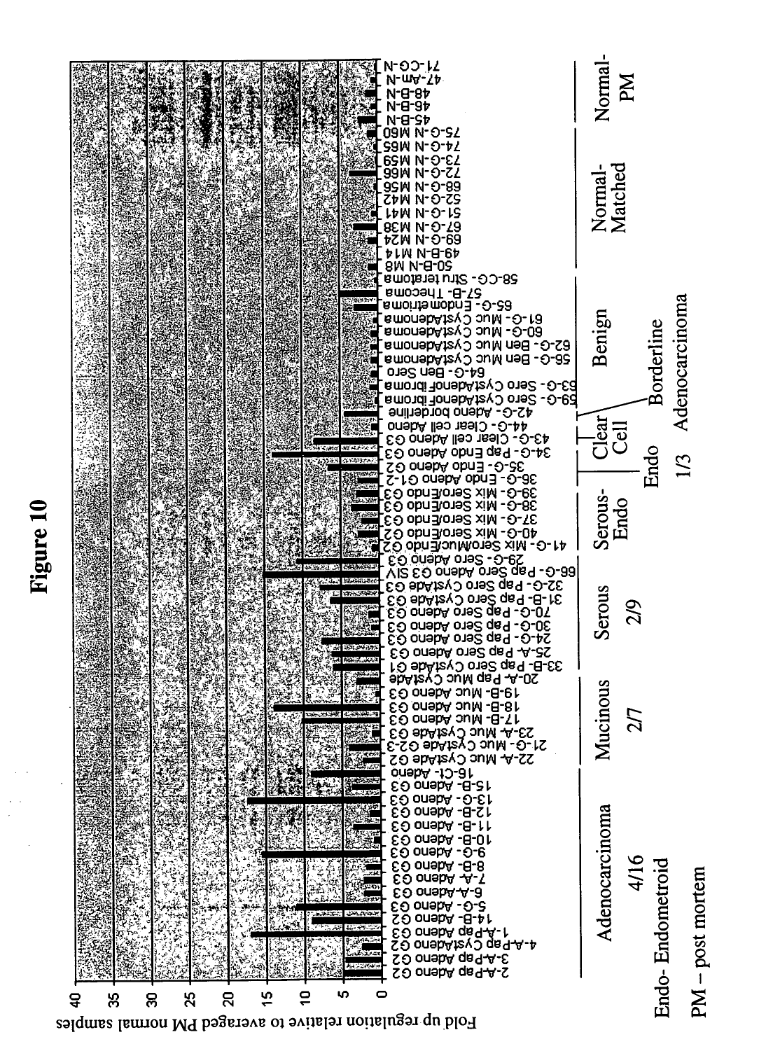

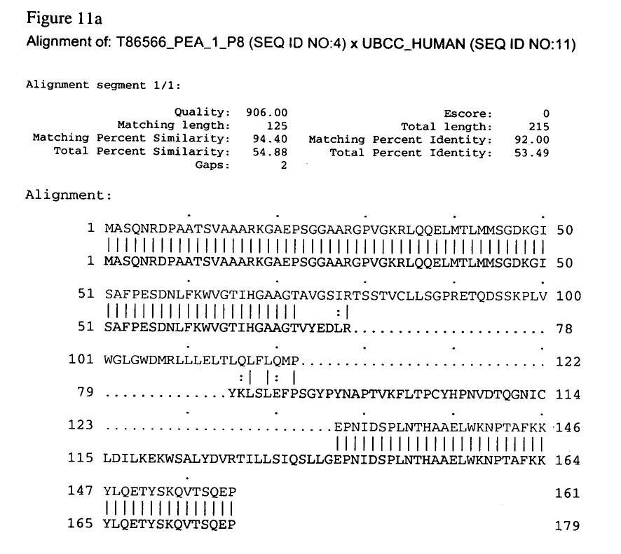

In FIG. 10, data is shown in a histogram presentation. The number of samples that exhibit at least 10 fold over-expression, out of the total number of samples tested is indicated below the cancer subtypes.

As shown in FIG. 10, over-expression of at least 10 fold was found for SEQ ID NO:14 in 4 out of 16 adenocacinoma, 2 out of 7 Mucinus adenocarcinoma, in 2 out of 9 Serous adenocarcinoma and 1 out of 3 endometroid adenocarcinoma samples.

According to the present invention, UbcH10 transcripts detectable by SEQ ID NO:14 (e.g., variant as set forth in SEQ ID NOs:1 and 2) are a non-limiting example of a marker for diagnosing ovarian cancer. The above UbcH10 variant marker of the present invention can be used alone or in combination, for various uses, including but not limited to, prognosis, prediction, screening, early diagnosis, staging, determination of cancer origin in organs which are different from ovary, therapy selection and treatment monitoring of ovarian cancer. Although optionally any method may be used to detect overexpression and/or differential expression of this marker, preferably a NAT-based technology is used. Therefore, optionally and preferably, any nucleic acid molecule capable of selectively hybridizing to UbcH10 transcripts detectable by SEQ ID NO:14 (e.g., variant as set forth in SEQ ID NOs:1 and 2) as previously defined is also encompassed within the present invention. Primer pairs are also optionally and preferably encompassed within the present invention; for example, for the above experiment, the following primer pair was used as a non-limiting illustrative example only of a suitable primer pair: SEQ ID NO:14 (amplicon of SEQ ID NOs:1 and 2); forward primer (SEQ ID NO:19): TCTACTGTCCGGTCCCAGAGA; and reverse primer (SEQ ID NO:20): AGTAAGCTCCAGCAGCAGCC.

The present invention also preferably encompasses any amplicon obtained through the use of any suitable primer pair; for example, for the above experiment, the following amplicon was obtained as a non-limiting illustrative example only of a suitable amplicon: amplicon (SEQ ID NO:14): TCTACTGTCCGGTCCCAG AGAAACTCAAGATTCTAGCAAGCCCCTTGTGTGGGGCTTGGGTTGGGACA TGAGGCTGCTGCTGGAGCTTAC

According to other preferred embodiments of the present invention, UbcH10 transcripts detectable by SEQ ID NO:14 (e.g., variant as set forth in SEQ ID NOs:1 and 2) or a fragment thereof comprises a biomarker for detecting ovarian cancer. Optionally and more preferably, the fragment of UbcH10 transcript detectable by SEQ ID NO:14 (e.g., variant as set forth in SEQ ID NOs:1 and 2) comprises segment_{—}19 (SEQ ID NO:9; which is contained in SEQ ID NOs:1 and 2). Optionally and more preferably, the fragment of UbcH10 transcript detectable by SEQ ID NO:14 (e.g., variant as set forth in SEQ ID NOs:1 and 2) comprises segment_{—}20 (SEQ ID NO:10, which is contained in SEQ ID NOs:1, 2 and 3). Also optionally and more preferably, any suitable method may be used for detecting a fragment such as segment_{—}19 for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to SEQ ID NO:1 as described above, including but not limited to SEQ ID NOs:4 and 7. According to still other preferred embodiments, the present invention optionally and preferably encompasses any amino acid sequence or fragment thereof encoded by a nucleic acid sequence corresponding to SEQ ID NO:2 as described above, including but not limited to SEQ ID NOs:5 and 7. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

The present invention also optionally and preferably encompasses any nucleic acid sequence or fragment thereof, or amino acid sequence or fragment thereof, corresponding to UbcH10 transcripts detectable by SEQ ID NO:14, as described above, optionally for any application.

Table 5 hereinbelow, summarizes the description of each of SEQ ID NO: used herein throughout the entire application.

TABLE 5

| SEQ ID | |
|---|---|
| NO | SEQUENCE DESCRIPTION |
| 1 | T86566_T8 (UbcH10 variant nucleic acid sequence) |
| 2 | T86566_T9 (UbcH10 variant nucleic acid sequence) |
| 3 | T86566_T19 (UbcH10 variant nucleic acid sequence) |
| 4 | Amino acid sequence of SEQ ID NO: 1 |
| 5 | Amino acid sequence of SEQ ID NO: 2 |
| 6 | Amino acid sequence of SEQ ID NO: 3 |
| 7 | Amino acid sequence coded by unique exons 4a, |
| | 4b of SEQ ID NOs: 1 and 2 |
| 8 | Amino acid sequence coded by unique exon |
| | 4b of SEQ ID NO: 3 |
| 9 | Segment 19; contained in SEQ ID NOs: 1 and 2 |
| 10 | segment 20; contained in SEQ ID NOs: 1, 2 and 3 |
| 11 | WT UbcH10 amino acid sequence (accession number O00762) |
| 12 | Amplicon of SEQ ID NO: 1 |
| 13 | Amplicon of SEQ ID NOs: 1 or 2 |
| 14 | Amplicon of SEQ ID NOs: 1 or 2 |
| 15 | Primer for SEQ ID NO: 12 |
| 16 | Primer for SEQ ID NO: 12 |
| 17 | Primer for SEQ ID NO: 13 |
| 18 | Primer for SEQ ID NO: 13 |
| 19 | Primer for SEQ ID NO: 14 |
| 20 | Primer for SEQ ID NO: 14 |
| 21 | PBGD amplicon |
| 22 | PBGD forward primer |
| 23 | PBGD reverse primer |
| 24 | HPRT1 amplicon |
| 25 | HPRT1 forward primer |
| 26 | HPRT1 reverse primer |
| 27 | GAPDH amplicon |
| 28 | GAPDH forward primer |
| 29 | GAPDH reverse primer |
| 30 | SDHA amplicon |
| 31 | SDHA forward primer |
| 32 | SDHA reverse primer |
| 33 | Ubiquitin amplicon |
| 34 | Ubiquitin forward primer |
| 35 | Ubiquitin reverse primer |
| 36 | WT UbcH10 (GenBank Accession No. U73379), |
| | nucleic acid sequence |
| 37 | Antisense of nucleic acids 219-238 of SEQ ID NO: 2 |
| 38 | Antisense of nucleic acids 307-326 of SEQ ID NO: 3 |
| 39 | Antisense of nucleic acids 409-429 of SEQ ID NO: 3 |
| 40 | siRNA of nucleic acids 309-329 of SEQ ID NO: 3 |
| 41 | siRNA of nucleic acids 411-429 of SEQ ID NO: 3 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. An isolated polynucleotide comprising a nucleic acid sequence encoding a UbcH10 polypeptide having at least a portion of an amino acid sequence at least 70% homologous to SEQ ID NO:7, as determined using the BlastP software of the National Center of Biotechnology Information (NCBI) using default parameters.

2. The isolated polynucleotide of claim 1, wherein said UbcH10 polypeptide is as set forth by SEQ ID NO:4.

3. The isolated polynucleotide of claim 1, wherein said nucleic acid sequence is as set forth in SEQ ID NO:1.

4. An isolated polypeptide encoding for UbcH10, comprising a first amino acid sequence being at least 90% homologous to amino acids 1-72 as set forth in SEQ ID NO:11, an edge polypeptide having an amino acid sequence at least 70% homologous to the amino acid sequence set forth by SEQ ID NO:7, and a second amino acid sequence being at least 90% homologous to amino acids 141-179 as set forth of SEQ ID NO:1, wherein said first amino acid is contiguous to said edge polypeptide and said second amino acid sequence is contiguous to said edge polypeptide, and wherein said first amino acid, said edge polypeptide and said second amino acid sequence are in a sequential order.

5. The isolated polypeptide of claim 4, wherein said isolated polypeptide is set forth by SEQ ID NO:4.

6. The isolated polypeptide of claim 5, wherein said edge polypeptide includes at least one bridge portion.

7. The isolated polypeptide of claim 6, wherein said at least one bridge portion includes a first bridge portion and a second bridge portion.

8. The isolated polypeptide of claim 7, wherein said first bridge portion comprises a polypeptide having “n” amino acids, wherein said “n” is at least 10 and whereas at least two amino acids of said first bridge portion are Threonine and Alanine, and wherein said first bridge portion has a structure as follows (numbering according to SEQ ID NO:4): a sequence starting from any of amino acid numbers 72−x to 72; and ending at any of amino acid numbers 73+((n−2)−x), in which x varies from 0 to n−2.

9. The isolated polypeptide of claim 7, wherein second bridge portion comprises a polypeptide having “n” amino acids, wherein said “n” is at least 10, and whereas at least two amino acids of said second bridge portion are Proline and Glutamic acid, and wherein said second bridge portion has a structure as follows (numbering according to SEQ ID NO:4): a sequence starting from any of amino acid numbers 122−x to 122; and ending at any of amino acid numbers 123+((n−2)−x), in which x varies from 0 to n−2.

10. The isolated polypeptide of claim 4, wherein said edge polypeptide is set forth by SEQ ID NO:7.

11. A method of diagnosing predisposition to, or presence of a UbcH10-related disease in a subject, the method comprising determining a level of a UbcH10 polypeptide including at least a portion of an amino acid sequence at least 70% homologous to a UbcH10 polypeptide as set forth in SEQ ID NO:4 as determined using the BlastP software of the National Center of Biotechnology Information (NCBI) using default parameters, or of a polynucleotide encoding said polypeptide in a biological sample obtained from the subject and being at least 70% identical to a polynucleotide as set forth by SEQ ID NO:1 wherein said level of said polynucleotide or said level of said polypeptide is correlatable with predisposition to, or presence or absence of the UbcH10-related disease, thereby diagnosing predisposition to, or presence of UbcH10-related disease in the subject.

12. The method of claim 11, wherein said determining level of said polypeptide is effected via an assay selected from the group consisting of immunohistochemistry, ELISA, RIA, Western blot analysis, FACS analysis, an immunofluorescence assay, and a light emission immunoassay.

13. The method of claim 11, wherein said determining level of said polynucleotide is effected via an assay selected from the group consisting of PCR, RT-PCR, quantitative RT-PCR, chip hybridization, RNase protection, in-situ hybridization, primer extension, Southern blot, Northern blot and dot blot analysis.

14. The method of claim 11, wherein the UbcH10-related disease is selected from the group consisting of ovarian cancer and lung cancer.


SEQUENCE LISTING (41 sequences)

SEQ ID NO: 1 - DNA, 783 bp, Homo sapiens
gcccccagagggttacaattcaaacgcgggcgggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacagcagtggggagcatcagaaccagctcaacagtttgtctactgtccggtcccagagaaactcaagattctagcaagccccttgtgtggggcttgggttgggacatgaggctgctgctggagcttactctgcaactgtttctccaaatgccagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttaaacaaaaaaat

SEQ ID NO: 2 - DNA, 696 bp, Homo sapiens
gcccccagagggttacaattcaaacgcgggcgggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatggcagtggggagcatcagaaccagctcaacagtttgtctactgtccggtcccagagaaactcaagattctagcaagccccttgtgtggggcttgggttgggacatgaggctgctgctggagcttactctgcaactgtttctccaaatgccagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttaaacaaaaaaat

SEQ ID NO: 3 - DNA, 935 bp, Homo sapiens
gcccccagagggttacaattcaaacgcgggcgggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacaagaaactcaagattctagcaagccccttgtgtggggcttgggttgggacatgaggctgctgctggagcttactctgcaactgtttctccaaatgccaggtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttaaacaaaaaaat

SEQ ID NO: 4 - protein, 161 aa, Homo sapiens
MASQNRDPAATSVAAARKGAEPSGGAARGPVGKRLQQELMTLMMSGDKGISAFPESDNLFKWVGTIHGAAGTAVGSIRTSSTVCLLSGPRETQDSSKPLVWGLGWDMRLLLELTLQLFLQMPEPNIDSPLNTHAAELWKNPTAFKKYLQETYSKQVTSQEP

SEQ ID NO: 5 - protein, 132 aa, Homo sapiens
MASQNRDPAATSVAAARKGAEPSGGAARGPVGKRLQQELMTLMAVGSIRTSSTVCLLSGPRETQDSSKPLVWGLGWDMRLLLELTLQLFLQMPEPNIDSPLNTHAAELWKNPTAFKKYLQETYSKQVTSQEP

SEQ ID NO: 6 - protein, 77 aa, Homo sapiens
MASQNRDPAATSVAAARKGAEPSGGAARGPVGKRLQQELMTLMMSGDKGISAFPESDNLFKWVGTIHGAAGTRNSRF

SEQ ID NO: 7 - protein, 50 aa, Homo sapiens
  misc_feature: Amino acid sequence encoded by unique exon 4a, 4b of the variants described in seq id no′s 1 and 2
AVGSIRTSSTVCLLSGPRETQDSSKPLVWGLGWDMRLLLELTLQLFLQMP

SEQ ID NO: 8 - protein, 5 aa, Homo sapiens
  misc_feature: Amino acid sequence encoded by unique exon 4b of the variant described in seq id no 1
RNSRF

SEQ ID NO: 9 - DNA, 53 bp, Homo sapiens
  misc_feature: Segment_19; contained in sequences described in SEQ ID NOs1 and 2
gcagtggggagcatcagaaccagctcaacagtttgtctactgtccggtcccag

SEQ ID NO: 10 - DNA, 98 bp, Homo sapiens
  misc_feature: Segment_20; contained in sequences described in SEQ ID NO′s 1, 2 and 3
agaaactcaagattctagcaagccccttgtgtggggcttgggttgggacatgaggctgctgctggagcttactctgcaactgtttctccaaatgccag

SEQ ID NO: 11 - protein, 179 aa, Homo sapiens
MASQNRDPAATSVAAARKGAEPSGGAARGPVGKRLQQELMTLMMSGDKGISAFPESDNLFKWVGTIHGAAGTVYEDLRYKLSLEFPSGYPYNAPTVKFLTPCYHPNVDTQGNICLDILKEKWSALYDVRTILLSIQSLLGEPNIDSPLNTHAAELWKNPTAFKKYLQETYSKQVTSQEP

SEQ ID NO: 12 - DNA, 103 bp, Artificial sequence
  PCR amplicon of SEQ ID 1
ttttcaaatgggtagggaccatccatggagcagctggaacagcagtggggagcatcagaaccagctcaacagtttgtctactgtccggtcccagagaaactca

SEQ ID NO: 13 - DNA, 126 bp, Artificial sequence
  PCR amplicon of SEQ ID′S 1 and 2
tgtttctccaaatgccagaacccaacattgatagtcccttgaacacacatgctgccgagctctggaaaaaccccacagcttttaagaagtacctgcaagaaacctactcaaagcaggtcaccagcc

SEQ ID NO: 14 - DNA, 90 bp, Artificial sequence
  PCR amplicon of SEQ ID′S 1 and 2
tctactgtccggtcccagagaaactcaagattctagcaagccccttgtgtggggcttgggttgggacatgaggctgctgctggagcttac

SEQ ID NO: 15 - DNA, 23 bp, Artificial sequence
  Single strand DNA oligonucleotide
ttttcaaatgggtagggaccatc

SEQ ID NO: 16 - DNA, 20 bp, Artificial sequence
  Single strand DNA oligonucleotide
tgagtttctctgggaccgga

SEQ ID NO: 17 - DNA, 22 bp, Artificial sequence
  Single strand DNA oligonucleotide
tgtttctccaaatgccagaacc

SEQ ID NO: 18 - DNA, 19 bp, Artificial sequence
  Single strand DNA oligonucleotide
ggctggtgacctgctttga

SEQ ID NO: 19 - DNA, 21 bp, Artificial sequence
  Single strand DNA oligonucleotide
tctactgtccggtcccagaga

SEQ ID NO: 20 - DNA, 20 bp, Artificial sequence
  Single strand DNA oligonucleotide
agtaagctccagcagcagcc

SEQ ID NO: 21 - DNA, 91 bp, Artificial sequence
  PBGD PCR amplicon
tgagagtgattcgcgtgggtacccgcaagagccagcttgctcgcatacagacggacagtgtggtggcaacattgaaagcctcgtaccctgg

SEQ ID NO: 22 - DNA, 19 bp, Artificial sequence
  Single strand DNA oligonucleotide
tgagagtgattcgcgtggg

SEQ ID NO: 23 - DNA, 21 bp, Artificial sequence
  Single strand DNA oligonucleotide
ccagggtacgaggctttcaat

SEQ ID NO: 24 - DNA, 94 bp, Artificial sequence
  HPRT1 PCR amplicon
tgacactggcaaaacaatgcagactttgctttccttggtcaggcagtataatccaaagatggtcaaggtcgcaagcttgctggtgaaaaggacc

SEQ ID NO: 25 - DNA, 21 bp, Artificial sequence
  Single strand DNA oligonucleotide
tgacactggcaaaacaatgca

SEQ ID NO: 26 - DNA, 21 bp, Artificial sequence
  Single strand DNA oligonucleotide
ggtccttttcaccagcaagct

SEQ ID NO: 27 - DNA, 116 bp, Artificial sequence
  GAPDH PCR amplicon
tgcaccaccaactgcttagcacccctggccaaggtcatccatgacaactttggtatcgtggaaggactcatgaccacagtccatgccatcactgccacccagaagactgtggatgg

SEQ ID NO: 28 - DNA, 20 bp, Artificial sequence
  Single strand DNA oligonucleotide
tgcaccaccaactgcttagc

SEQ ID NO: 29 - DNA, 19 bp, Artificial sequence
  Single strand DNA oligonucleotide
ccatcacgccacagtttcc

SEQ ID NO: 30 - DNA, 86 bp, Artificial sequence
  SDHA PCR amplicon
tgggaacaagagggcatctgctaaagtttcagattccatttctgctcagtatccagtagtggatcatgaatttgatgcagtggtgg

SEQ ID NO: 31 - DNA, 20 bp, Artificial sequence
  Single strand DNA oligonucleotide
tgggaacaagagggcatctg

SEQ ID NO: 32 - DNA, 22 bp, Artificial sequence
  Single strand DNA oligonucleotide
ccaccactgcatcaaattcatg

SEQ ID NO: 33 - DNA, 133 bp, Artificial sequence
  Ubiquitin amplicon
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 34 - DNA, 19 bp, Artificial sequence
  Single strand DNA oligonucleotide
atttgggtcgcggttcttg

SEQ ID NO: 35 - DNA, 21 bp, Artificial sequence
  Single strand DNA oligonucleotide
tgccttgacattctcgatggt

SEQ ID NO: 36 - DNA, 809 bp, Homo sapiens
gggcgggcccgcagtcctgcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacagtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtccttttttaaaaaaaaaaaaaaa

SEQ ID NO: 37 - DNA, 20 bp, Artificial sequence
  Antisense oligonucleotide complementary to the Variant described in SEQ ID NO 2
cccactgccatgagggtcat

SEQ ID NO: 38 - DNA, 20 bp, Artificial sequence
  Antisense oligonucleotide complementary to the Variant described in SEQ ID NO 3
tgagtttcttgttccagctg

SEQ ID NO: 39 - DNA, 20 bp, Artificial sequence
  Antisense oligonucleotide complementary to the Variant described in SEQ ID NO 3
ggtcttcatatacctggcat

SEQ ID NO: 40 - DNA, 21 bp, Artificial sequence
  SiRNA oligonucleotide targeting the Variant described in SEQ ID NO 3
gctggaacaagaaactcaaga

SEQ ID NO: 41 - DNA, 19 bp, Artificial sequence
  SiRNA oligonucleotide targeting the Variant described in SEQ ID NO 3
gccaggtatatgaagacct